(19) United States
(12) Patent Application Publication
(10) Pub. No.: US 2024/0197780 A1
(43) Pub. Date: Jun. 20, 2024

(54) CHIMERIC ANTIGEN RECEPTORS, NUCLEIC ACIDS ENCODING THE SAME, AND USES THEREOF IN TREATING CANCERS

(71) Applicant: Development Center for Biotechnology, New Taipei City (TW)

(72) Inventors: Li-Shuang AI, Taipei (TW); Yu-Hsun LO, Taipei (TW); Cheng-Chou YU, Taipei (TW); Yi-Jiue TSAI, Taipei (TW); Hsin-Yi TSAI, Taipei (TW); Show-Shan SHEU, Taipei (TW); Yi-Tian HE, 115 (TW); Pei-Yi TSAI, Taipei (TW); Chia-Tsen LAI, Taipei (TW)

(21) Appl. No.: 18/542,884

(22) Filed: Dec. 18, 2023

Related U.S. Application Data
(60) Provisional application No. 63/433,503, filed on Dec. 19, 2022.

(51) Int. Cl. A61K 35/17; A61K 39/00; A61P 35/00; C07K 14/705; C07K 16/28

(52) U.S. Cl. CPC A61K 35/17; A61K 39/4611; A61K 39/4631; A61K 39/464411; A61K 39/464429; A61P 35/00; C07K 14/70503; C07K 14/70596; C07K 16/2818; C07K 16/2827; A61K 2239/15; A61K 2239/17; A61K 2239/21; A61K 2239/29

(57) ABSTRACT

Disclosed herein is a chimeric antigen receptor (CAR) comprising a single-chain variable fragment specific to Globo H, a hinge and transmembrane domain, a co-stimulatory molecule, and a cytoplasmic domain. According to some embodiments of the present disclosure, the CAR further comprises a single-chain variable fragment specific to PD-L1, and optionally, a fragment crystallizable region of an immunoglobulin. Also disclosed herein are isolated nucleic acids encoding the CAR pharmaceutical kits comprising the isolated immune cells expressing the CAR, and methods of treating cancers by using isolated immune cells.

### CROSS-REFERENCE TO RELATED APPLICATIONS

This application relates to and claims the benefit of U.S. Provisional Application No. 63/433,503, filed Dec. 19, 2022; the content of the application is incorporated herein by reference in its entirety.

### SEQUENCE LISTING XML

The present application is being filed along with a Sequence Listing XML in electronic format. The Sequence Listing XML is provided as an XML file entitled P4306_SEQ_AF, created Dec. 15, 2023, which is 67 Kb in size. The information in the electronic format of the Sequence Listing XML is incorporated herein by reference in its entirety.

### BACKGROUND OF THE INVENTION

### 1. Field of the Invention

The present disclosure in general relates to the field of disease treatment. More particularly, the present disclosure relates to the treatment of cancers by use of novel chimeric antigen receptor T (CAR-T) cells.

### 2. Description of Related Art

Globohexaosylceramide (Globo H; Fuc-α1,2-Gal-β1,3-GalNAc-β1,3-Gal-α1,4-Gal-β1,4-Glc-β1,1-Cer) is a glycosphingolipid antigen consisting of a hexasaccharide linked to a ceramide. It is known that Globo H is highly expressed on the surface of cancer stem cells and various types of cancer cells, including lung, breast, prostate, gastric, ovarian, colon-rectum, pancreatic, liver, and uterine cancer cells. Further, Globo H is known to be associated with angiogenesis and immunosuppress through Notch signaling. Compared to the cells expressing low levels of Globo H, the cells expressing high levels of Globo H exhibit greater tumorigenicity and angiogenicity. The high Globo H expression by cancer and cancer stem cells made it an attractive target for the development of immunotherapeutic agents against various cancers.

CAR-T cells are T cells that have been genetically engineered to express an artificial T cell receptor on their surfaces. In general, the CAR at least comprises three domains, including an antigen recognition domain, a transmembrane domain and an intracellular signaling domain. The antigen recognition domain is exposed to the outside of the cell (i.e., an ectodomain portion of the receptor), and is configured to interact with a potential target molecule (e.g., tumor-associated antigen, TAA) for targeting the CAR-T cell to the cells expressing the target molecule (e.g., cancer cells). The transmembrane domain anchors the CAR to the plasma membrane of the cell, and bridges the extracellular antigen recognition domain with the intracellular signaling domain. The transmembrane domain is essential for the stability of the CAR as a whole. Regarding the intracellular signaling domain, it is located in the endodomain of the cell accounting for intracellular signaling and mediating the activation of CAR-expressing T cell. Although the treatment with CAR-T cells has produced remarkable clinical responses with certain subsets of B cell leukemia or lymphoma, the application of CAR-T cells in solid tumors is still limited due to immunosuppressive tumor microenvironment (TME) that compromises T cell function.

In view of the foregoing, there is a continuing interest in developing a novel CAR-T cell that is resistant to immunosuppression and/or is capable of overcoming immunosuppression thereby improving the therapeutic efficacy on solid tumors.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the first aspect of the disclosure is directed to a chimeric antigen receptor (CAR), which comprises in sequence, from N-terminus to C-terminus,
(a) a first single-chain variable fragment (scFv) specifically binds to Globo H;
(b) a hinge and transmembrane (HTM) domain;
(c) a co-stimulatory molecule; and
(d) a cytoplasmic domain.

The first scFv, in its structure, comprises a heavy chain variable (VH) domain and a light chain variable (VL) domain, in which the VH domain comprises a first heavy chain complementarity determining region (CDR-H1), a second heavy chain CDR (CDR-H2) and a third heavy chain CDR (CDR-H3), and the VL domain comprises a first light chain CDR (CDR-L1), a second light chain CDR (CDR-L2) and a third light chain CDR (CDR-L3). According to some embodiments of the present disclosure, the CDR-H1, CDR-H2 and CDR-H3 respectively comprise the amino acid sequences of SEQ ID NOs: 1-3, and CDR-L1, CDR-L2 and CDR-L3 respectively comprise the amino acid sequences of SEQ ID NOs: 4-6. In some preferred embodiments, the VH and VL domains of the Globo H-specific scFv respectively comprise the amino acid sequences of SEQ ID NOs: 7 and 8.

According to some embodiments of the present disclosure, the HTM domain is a HTM domain of differentiation 8 (CD8), the co-stimulatory molecule is 4-1BB, and the cytoplasmic domain is a cytoplasmic domain of CD3 zeta chain (CD3ζ). In certain exemplary embodiments, the HTM domain of CD8, the 4-1BB co-stimulatory molecule, and the cytoplasmic domain of CD3ζ respectively comprise the amino acid sequences of SEQ ID NOs: 9-11.

According to certain preferred embodiments of the present disclosure, the CAR further comprises,
(e) a second scFv specifically binds to programmed death-ligand 1 (PD-L1); and
(f) a linker linking the second scFv to the C-terminus of the cytoplasmic domain, wherein the linker is a 2A peptide or an internal ribosomal entry site (IRES) element.

The second scFv, in its structure, comprises a VH domain and a VL domain, in which the VH domain comprises a CDR-H1, a CDR-H2 and a CDR-H3, and the VL domain comprises a CDR-L1, a CDR-L2 and a CDR-L3. According to certain embodiments, the CDR-H1, CDR-H2 and CDR-H3 of the PD-L1-specific scFv respectively comprise the amino acid sequences of SEQ ID NOs: 12-14, and the CDR-L1, CDR-L2 and CDR-L3 of the PD-L1-specific scFv respectively comprise the amino acid sequences of SEQ ID NOs: 15-17. Preferably, the VH and VL domains of the PD-L1-specific scFv respectively comprise the amino acid sequences of SEQ ID NOs: 18 and 19. According to some alternative embodiments, the CDR-H1, CDR-H2 and CDR-H3 of the PD-L1-specific scFv respectively comprise the amino acid sequences of SEQ ID NOs: 20-22, and the CDR-L1, CDR-L2 and CDR-L3 of the PD-L1-specific scFv respectively comprise the amino acid sequences of SEQ ID NOs: 23-25. Preferably, the VH and VL domains of the PD-L1-specific scFv respectively comprise the amino acid sequences of SEQ ID NOs: 26 and 27.

Optionally, the CAR further comprises,
(g) a fragment crystallizable region (Fc region) of an immunoglobulin that is disposed at the C-terminus of the second scFv.

Depending on desired purpose, the immunoglobulin may be an immunoglobulin G (IgG), immunoglobulin A (IgA), immunoglobulin M (IgM), immunoglobulin D (IgD) or immunoglobulin E (IgE). According to one exemplary embodiment, the immunoglobulin is IgG. In the embodiment, the present CAR comprises the amino acid sequence of SEQ ID NO: 28 or 29. According to some alternatively embodiments, the PD-L1-specific scFv is an humanized antibody; in these embodiments, the CAR comprises the amino acid sequence of SEQ ID NO: 30, 31, 32 or 33.

Also disclosed therein are an isolated nucleic acid encoding the CAR of the present disclosure, and an expression vector comprising the isolated nucleic acid.

According to some embodiments of the present disclosure, the isolated nucleic acid comprises in sequence, from 5′ end to 3′ end, a first, a second, a third and a fourth coding sequences, which respectively encode the first scFv, HTM domain, co-stimulatory molecule and cytoplasmic domain of the CAR. In these embodiments, the first, second, third and fourth coding sequences respectively comprise the nucleotide sequences at least 85% identical to SEQ ID NOs: 38, 39, 40 and 41. In some examples, the first, second, third and fourth coding sequences respectively comprise the nucleotide sequences 100% identical to SEQ ID NOs: 38, 39, 40 and 41, i.e., respectively comprising the nucleotide sequences of SEQ ID NOs: 38, 39, 40 and 41.

According to some embodiments of the present disclosure, in addition to the first, second, third and fourth coding sequences, the isolated nucleic acid further comprises a linker sequence and a fifth coding sequence, wherein the fifth coding sequence is linked to the 3′ end of the fourth coding sequence via the linker sequence. In these embodiments, the linker sequence is an IRES or encodes a 2A peptide, and the fifth coding sequence encodes a second scFv specific to PD-L1. According to some examples, the fifth coding sequence comprises a nucleotide sequence at least 85% identical to SEQ ID NO: 43 or 44. In one specific example, the fifth coding sequence comprises a nucleotide sequence 100% identical to SEQ ID NO: 43 or 44, i.e., comprising the nucleotide sequence of SEQ ID NO: 43 or 44.

According to certain embodiments of the present disclosure, in addition to the first, second, third, fourth and fifth coding sequences and the linker sequence, the isolated nucleic acid further comprises a sixth coding sequence linked to the 3′ end of the fifth coding sequence. In these embodiments, the sixth coding sequence encodes a fragment crystallizable region (Fc region) of an immunoglobulin, and comprises a nucleotide sequence at least 85% identical to SEQ ID NO: 47. In one specific example, the sixth coding sequence comprises a nucleotide sequence 100% identical to SEQ ID NO: 47, i.e., comprising the nucleotide sequence of SEQ ID NO: 47.

According to certain embodiments, the expression vector comprising the isolated nucleic acid is a viral vector; for example, a lentiviral vector, an adenoviral vector, a retroviral vector, an adeno-associated viral vector, or a sindbis viral vector. In one exemplary embodiment, the expression vector is the lentiviral vector.

Another aspect of the present disclosure pertains to the use of the isolated nucleic acid for the preparation of a genetically modified cell, and uses thereof in the treatment of cancers.

According to some embodiments of the present disclosure, the genetically modified cell comprises the isolated nucleic acid and expresses the present CAR on its surface. Preferably, the genetically modified cell is a genetically modified immune cell, such as a genetically modified T cell, a genetically modified natural killer (NK) cell, or a genetically modified macrophage.

The genetically modified immune cell is useful in treating cancers via recognizing and specifically binding to the cancers through the CAR. Accordingly, also disclosed herein is a method of treating a cancer in a subject. The method comprises administering to the subject an effective amount of the genetically modified immune cell, so as to alleviate or ameliorate the symptoms of the cancer.

Depending on intended purpose, the cancer may be gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma. Preferably, the cancer is a Globo H-positive cancer (i.e., a cancer expressing Globo H).

The subject treatable with the genetically modified immune cell and/or method of the present disclosure is a mammal; preferably, a human.

The present disclosure also provides a pharmaceutical kit for the treatment of cancers. According to some embodiments of the present disclosure, the pharmaceutical kit comprises the present genetically modified immune cell, and an inhibitor of PD-L1 or an inhibitor of programmed death-ligand 1 (PD-L1).

According to some preferred embodiments of the present disclosure, the inhibitor of PD-L1 is Atezolizumab, and the inhibitor of programmed death 1 (PD-1) is Nivolumab.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings briefly discussed below.

FIG. 1 depicts the schematic diagrams of the present CAR according to the embodiments of the present disclosure; Panel (A): Globo H CAR; Panel (B): Globo H/PD-L1 scFv CAR; Panel (C): Globo H/PD-L1 scFv-Fc CAR. CD8 HTM: The hinge and transmembrane domain of CD8. CD3ζ CD: The cytoplasmic domain of CD3ζ.

FIG. 2 depicts the anti-tumor effect of specified CAR-T cells according to Example 4.1 of the present disclosure; Panel (A): Cytotoxicity of specified CAR-T cells; Panel (B): Expression level of IFN-γ of specified CAR-T cells; Panel (C): Expression level of PD-L1 of specified CAR-T cells. Globo H: Globo H CAR-T cell. Globo H/PD-L1^{Ate-Fc1}: Globo H/PD-L1^{Ate} scFv-Fc CAR-T cell. Globo H/PD-L1^{1G8-Fc1}: Globo H/PD-L1^{1G8} scFv-Fc CAR-T cell. Globo H/PD-L1^{3C3-Fc1:} Globo H/PD-L1^{3C3} scFv-Fc CAR-T cell. FMC63-GFP: T cells expressing anti-CD19 scFv and green fluorescent protein (GFP); serving as a control group. Kappa-GFP: T cells expressing GFP; serving as another control group.

FIG. 3 depicts the therapeutic effect of specified CAR-T cells on cancers according to Example 4.2 of the present disclosure. GH: Globo H CAR-T cell. GH/PD-1^{Nivo-Fc1}: Globo H CAR/Nivo scFv-Fc CAR-T cell. GH/PD-L1^{Ate-Fc1:} Globo H/PD-L1^{Ate} scFv-Fc CAR-T cell. Globo H/PD-L1^{1G8-Fc1:} Globo H/PD-L1^{1G8} scFv-Fc CAR-T cell. Globo H/PD-L1^{3C3-Fc1:} Globo H/PD-L1^{3C3} scFv-Fc CAR-T cell.

In accordance with common practice, the various described features/elements are not drawn to scale but instead are drawn to best illustrate specific features/elements relevant to the present invention. Also, like reference numerals and designations in the various drawings are used to indicate like elements/parts.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “nucleic acid” refers to a polynucleotide such as deoxyribonucleic acid (DNA) and where appropriate, ribonucleic acid (RNA). Nucleic acids include but are not limited to single-stranded and double-stranded polynucleotides. Illustrative polynucleotides include DNA, single-stranded DNA, cDNA, and mRNA. The term also includes, analogs of either DNA or RNA made from nucleotide analogs, and as applicable, single (sense or antisense) and double-stranded polynucleotides. The term further includes modified polynucleotides, including modified DNA and modified RNA, e.g., DNA and RNA comprising one or more unnatural nucleotide or nucleoside. The terms “nucleic acid” is used herein to refer to deoxyribonucleotides or ribonucleotides and polymers thereof in either single-or double-stranded form. The term encompasses nucleic acids containing known nucleotide analogs or modified backbone residues or linkages, which are synthetic, naturally occurring, and non-naturally occurring, and/or which have similar binding properties as the reference nucleic acid, and/or which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “antibody” (Ab) is used in its meaning known in the art of cell biology and biochemistry, and covers monoclonal antibodies (including full length monoclonal antibodies), polyclonal antibodies, multi-specific or multivalent antibodies (e.g., bi-specific antibodies), chimeric antibodies, humanized antibodies and antibody fragments so long as they exhibit the desired biological activity. The term “antibody fragment” may be produced by recombinant DNA techniques or by enzymatic or chemical cleavage of intact antibodies. Examples of the antibody fragment include, fragment antigen-binding (Fab), Fab′, F(ab′)₂, single-chain variable fragment (scFv), domain antibody (dAb), diabodies, triabodies, tetrabodies, and polypeptides that contain at least a portion of an immunoglobulin that is sufficient to confer specific antigen binding to the polypeptide.

The term “single-chain variable fragment” (scFv) is used in its meaning known in the art of cell biology and biochemistry, and refers to a fusion protein of the variable domains of the heavy chain (VH) and light chain (VL) of an immunoglobulin, linked together with a short (usually serine and/or glycine) linker peptide. The scFv retains the specificity of the original immunoglobulin, despite removal of the constant domains and the introduction of the linker.

The term “complementarity determining region” (CDR) used herein refers to the hypervariable region of an antibody molecule that forms a surface complementary to the three-dimensional surface of a bound antigen. Proceeding from N-terminus to C-terminus, each of the antibody heavy and light chains comprises three CDRs (CDR-1, CDR-2 and CDR-3). An antigen combining site, therefore, includes a total of six CDRs that comprise three CDRs in the variable domain of a heavy chain (i.e., CDR-H1, CDR-H2 and CDR-H3), and three CDRs in the variable domain of a light chain (i.e., CDR-L1, CDR-L2 and CDR-L3).

The “variable domain” of an antibody refers to the amino-terminal domains of heavy or light chain of the antibody. These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in cach chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)).

As discussed herein, minor variations in the amino acid sequences of antibodies are contemplated as being encompassed by the presently disclosed and claimed inventive concept(s), providing that the variations in the amino acid sequence maintain at least 85% sequence identity, such as at least 85%, 86%, 87%, 88%, 89%, 90%, 91%, 92%, 93%, 94%, 95%, 96%, 97%, 98% and 99% sequence identity. The antibody of the present disclosure may be modified specifically to alter a feature of the antibody unrelated to its physiological activity. For example, certain amino acid residues in the framework (FR) region of the antibody can be changed and/or deleted without affecting the physiological activity of the antibody in this study (i.e., its ability to treat cancers). In particular, conservative amino acid replacements are contemplated. Conservative replacements are those that take place within a family of amino acid residues that are related in their side chains. Genetically encoded amino acid residues are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid residue within the antigen-biding sites, i.e., CDRs. Whether an amino acid change results in a functional peptide can readily be determined by assaying the specific activity of the peptide derivative. Fragments or analogs of proteins/peptides can be readily prepared by those of ordinary skill in the art. Preferred amino-and carboxy-termini of fragments or analogs occur near boundaries of functional domains.

“Percentage (%) sequence identity” is defined as the percentage of amino acid residues in a candidate sequence that are identical with the amino acid residues in the specific peptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100$$

where X is the number of amino acid residues scored as identical matches by the sequence alignment program BLAST in that program's alignment of A and B, and where Y is the total number of amino acid residues in A or B, whichever is shorter.

As used herein, the term “link” refers to any means of connecting two components either via direct linkage or via indirect linkage between two components.

As used herein, the term “treat,” “treating” and “treatment” are interchangeable, and encompasses partially or completely preventing, ameliorating, mitigating and/or managing a symptom, a secondary disorder or a condition associated with cancers. The term “treating” as used herein refers to application or administration of the antibody of the present disclosure to a subject, who has a symptom, a secondary disorder or a condition associated with cancers, with the purpose to partially or completely alleviate, ameliorate, relieve, delay onset of, inhibit progression of, reduce severity of, and/or reduce incidence of one or more symptoms, secondary disorders or features associated with cancers. Symptoms, secondary disorders, and/or conditions associated with cancers include, but are not limited to, nausea, vomiting, loss of appetite, constipation, fatigue, muscle weakness, increased thirst, bone pain or broken bones, swelling or lump, blooding, cough, fever, night sweats, coma and pain. Treatment may be administered to a subject who exhibits only early signs of such symptoms, disorder, and/or condition for the purpose of decreasing the risk of developing the symptoms, secondary disorders, and/or conditions associated with cancers. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, as grams, milligrams or micrograms; as milligrams per kilogram of body weight (mg/Kg); or as cell numbers of body weight (cells/Kg). Persons having ordinary skills could calculate the human equivalent dose (HED) for the medicament (such as the present CAR-T cells) based on the doses determined from animal models. For example, one may follow the guidance for industry published by U.S. Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The terms “subject” refers to an animal including the human species that is treatable by the CAR-T cells and/or method of the present invention. The term “subject” is intended to refer to both the male and female gender unless one gender is specifically indicated.

### II. Description of the Invention

The present disclosure aims at providing a novel CAR and a nucleic acid encoding the CAR. After introducing the nucleic acid into an immune cell (i.e., T cell), the CAR would be expressed on the surface of the immune cell. The thus-produced CAR-expressing immune cell (e.g., CAR-T cell) not only exhibits a binding specificity and cytotoxicity toward cancer cells, but also possesses an inhibitory effect on immunosuppressive factor PD-L1 present in tumor microenvironment. The inhibitory effect on PD-L1 results in enhanced anti-tumor response of the CAR-T cell in solid tumors. Accordingly, also disclosed herein are the CAR-expressing immune cell (e.g., CAR-T cell), and uses of the cell in treating cancers.

### (i) CAR and Isolated Nucleic Acid Encoding the Same

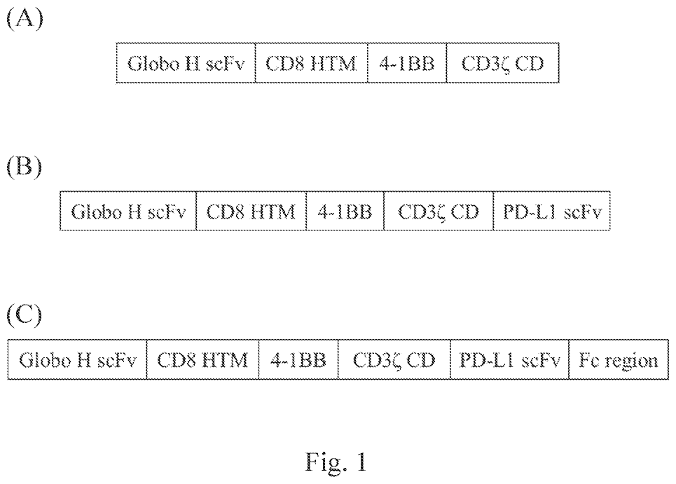

The first aspect of present disclosure is directed to the CAR. Reference is made to Panel (A) of FIG. 1, which is a schematic diagram of the present CAR according to some embodiments of the present disclosure. As depicted in Panel (A) of FIG. 1, the present CAR (designated as “Globo H CAR”) comprises in sequence, from N-terminus to C-terminus,
(a) an scFv specific to Globo H (i.e., an anti-Globo H scFv);
(b) a hinge and transmembrane domain of CD8 (i.e., CD8 HTM domain);
(c) a 4-1BB co-stimulatory molecule; and
(d) a cytoplasmic domain of CD3ζ.

In structure, the anti-Globo H scFv comprises three CDRs in the VH domain thereof (i.e., CDR-H1, CDR-H2, and CDR-H3), and three CDRs in the VL domain thereof (i.e., CDR-L1, CDR-L2, and CDR-L3). According to some preferred embodiments, the CDR-H1, CDR-H2, and CDR-H3 of the anti-Globo H scFv respectively comprise the amino acid sequences of SEQ ID NOs: 1-3; and the CDR-L1, CDR-L2, and CDR-L3 of the anti-Globo H scFv respectively comprise the amino acid sequences of SEQ ID NOs: 4-6. In one specific embodiment, the VH and VL domains of the anti-Globo H scFv respectively comprise the amino acid sequences of SEQ ID NOs: 7 and 8.

Since the binding affinity and specificity of an antibody are mainly determined by the CDR sequences thereof, as could be appreciated, the framework (FR) sequences of the VH and VL domains may vary (e.g., being substituted by conserved or non-conserved amino acid residues) without affecting the binding affinity and/or specificity of the present scFv. Preferably, the FR sequence is conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue).

Based on the conservative substitution, a skilled artisan may substitute the amino acid residue(s) of the FR sequences of the VH and VL domains of the anti-Globo H scFv without affecting the activity and/or effect of the anti-Globo H scFv (i.e., recognizing and targeting cancer cells). Accordingly, the antibody comprising substituted amino acid(s) in its FR sequences of VH and VL domains are intended to be included within the scope of the present disclosure. According to certain embodiments, the VH domain of the anti-Globo H scFv comprises the amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 7, and the VL domain of the anti-Globo H scFv comprises the amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 8. According to some preferred embodiments, the VH and VL domains of the anti-Globo H scFv respectively comprise the amino acid sequences at least 90% identical to SEQ ID NOs: 7 and 8. More preferably, the VH and VL domains of the anti-Globo H scFv respectively comprise the amino acid sequences at least 95% identical to SEQ ID NOs: 7 and 8.

According to some embodiments, the CD8 HTM domain comprises the amino acid sequence of SEQ ID NO: 9; the 4-1BB co-stimulatory molecule comprises the amino acid sequence of SEQ ID NO: 10; and the cytoplasmic domain of CD3ζ comprises the amino acid sequence of SEQ ID NO: 11.

Depending on desired purpose, the hinge domain of the CD8 HTM domain may alternatively be derived from CD28, IgG1 or IgG4, for example, the hinge domain of CD28, IgG1 or IgG4; and/or the transmembrane domain of the CD8 HTM domain may alternatively be derived from CD3 zeta chain (CD3ζ), CD8 alpha chain (CD8α), CD4, CD28 or B7-family inducible costimulator (ICOS), for example, the transmembrane domain of CD3ζ, CD8ζ, CD4, CD28 or ICOS.

As could be appreciated, in addition of the 4-1BB molecule, the present CAR may further comprises another co-stimulatory molecules, such as CD27, CD28 or OX40 (CD134). Alternatively, the 4-1BB molecule of the present CAR may be substituted by other co-stimulatory molecules, such as CD27, CD28 or OX40 (CD134).

For the purpose of overcoming the immunosuppressive factor present in tumor microenvironment, the present CAR preferably further includes a molecule against the immunosuppressive factor. Reference is now made to Panel (B) of FIG. 1, which is a schematic diagram of the present CAR (designated as “Globo H/PD-L1 scFv CAR”) according to certain embodiments of the present disclosure. Compared to the Globo H CAR depicted in Panel (A) of FIG. 1, the Globo H/PD-L1 scFv CAR of Panel (B) of FIG. 1 further comprises,
(e) an scFv specific to PD-L1 (i.e., an anti-PD-L1 scFv); and
(f) a linker linking the anti-PD-L1 scFv to the C-terminus of the cytoplasmic domain of CD3ζ.

In these embodiments, the Globo H/PD-L1 scFv CAR comprises in sequence, from N-terminus to C-terminus, an anti-Globo H scFv, a CD8 HTM domain, a 4-1BB co-stimulatory molecule, a cytoplasmic domain of CD3ζ, a linker, and an anti-PD-L1 scFv.

Preferably, the Globo H/PD-L1 scFv CAR further comprises a signal peptide (also known as “signal sequence” or “leader sequence”) disposed between the linker and the anti-PD-L1 scFv. As known in the art, the signal peptide refers to a peptide having about 15-50 amino acid residues in length that directs proteins toward secretory pathway. Examples of the signal peptide suitable to use in the present Globo H/PD-L1 scFv CAR include, but are not limited to, the signal peptide of tissue plasminogen activator (tPA), IgK, IgG, CD33, metalloproteinase inhibitor 1 (TIMP1), chronodroitin sulphate protcoglycan 4 (CSPG4), calreticulin (CALR), dickkopf-related protein 3 (DKK3), 60S acidic ribosomal protein P2 (RPLP2), complement C1s (C1S), cathepsin Z (CTSZ), nucleobinin-2 (NUCB2), protein disulphide-isomerase (PDIA1), protein disulphide-isomerase A3 (PDIA3), endoplasmin, hypoxia upregulated protein 1 (HYOU1), trypsinogen-2, serum albumin, and serpinh1. According to one exemplary embodiment of the present disclosure, the signal peptide is derived from light chain of IgG. A skilled artisan may choose a suitable signal peptide in accordance with practical needs.

As an example, two anti-PD-L1 scFvs respectively designated as “1G8” and “3C3” are provided in the present disclosure, in which each of the anti-PD-L1 scFvs comprises three CDRs in the VH domain thereof (i.e., CDR-H1, CDR-H2, and CDR-H3), and three CDRs in the VL domain thereof (i.e., CDR-L1, CDR-L2, and CDR-L3).

According to some embodiments, the CDR-H1, CDR-H2, and CDR-H3 of the scFv 1G8 respectively comprise the amino acid sequences of SEQ ID NOs: 12-14; and the CDR-L1, CDR-L2, and CDR-L3 of the scFv 1G8 respectively comprise the amino acid sequences of SEQ ID NOs: 15-17. In one specific embodiment, the VH and VL domains of the scFv 1G8 respectively comprise the amino acid sequences of SEQ ID NOs: 18 and 19.

According to alternative embodiments, the CDR-H1, CDR-H2, and CDR-H3 of the scFv 3C3 respectively comprise the amino acid sequences of SEQ ID NOs: 20-22; and the CDR-L1, CDR-L2, and CDR-L3 of the scFv 3C3 respectively comprise the amino acid sequences of SEQ ID NOs: 23-25. In one specific embodiment, the VH and VL domains of the scFv 3C3 respectively comprise the amino acid sequences of SEQ ID NOs: 26 and 27.

As described above, the FR sequences of the VH and VL domains may vary (e.g., being substituted by conserved or non-conserved amino acid residues) without affecting the binding affinity and/or specificity of the present scFv, and a skilled artisan may substitute the amino acid residue(s) of the FR sequences of the VH and VL domains of scFv 1G8 or 3C3 without affecting its activity and/or effect (i.e., inhibiting the activity of PD-L1). Accordingly, the antibody comprising substituted amino acid(s) in its FR sequences of VH and VL domains are intended to be included within the scope of the present disclosure. According to certain embodiments, the VH and VL domains of scFv 1G8 respectively comprise the amino acid sequences at least 85% identical to SEQ ID NOs: 18 and 19; preferably, at least 90% identical to SEQ ID NOs: 18 and 19; more preferably, at least 95% identical to SEQ ID NOs: 18 and 19. According to certain embodiments, the VH and VL domains of scFv 3C3 respectively comprise the amino acid sequences at least 85% identical to SEQ ID NOs: 26 and 27; preferably, at least 90% identical to SEQ ID NOs: 26 and 27; more preferably, at least 95% identical to SEQ ID NOs: 26 and 27.

Regarding the linker, it may be a 2A peptide, an IRES, or any peptides or sequence known to express multiple proteins form one transcript. 2A peptide also known as “2A self-cleaving peptide” is a class of peptide having 18 to 22 amino acid residues in length, which can induce ribosomal skipping during translation of a protein in cells. Examples of 2A peptide commonly used in the art include, but are not limited to, T2A (EGRGSLLTCGDVEENPGP; SEQ ID NO: 34), P2A (ATNFSLLKQAGDVEENPGP; SEQ ID NO: 35), E2A (QCTNYALLKLAGDVESNPGP; SEQ ID NO: 36) and F2A (VKQTLNFDLLKLAGDVESNPGP; SEQ ID NO: 37). IRES is a sequence that recruits ribosomes and allows cap-independent translation. In practice, IRES serves as a linker linking two coding sequences in one bicistronic vector and allowing the translation of both proteins in cells. According to some exemplary embodiments, the linker for linking the present anti-PD-L1 scFv and the cytoplasmic domain of CD3ζ is a T2A peptide.

Preferably, the present CAR further includes an Fc region of an immunoglobulin. Reference is made to Panel (C) of FIG. 1, which is a schematic diagram of the present CAR (designated as “Globo H/PD-L1 scFv-Fc CAR”) according to certain embodiments of the present disclosure. Compared to the Globo H/PD-L1 scFv CAR depicted in Panel (B) of FIG. 1, the Globo H/PD-L1 scFv-Fc CAR of Panel (C) of FIG. 1 further comprises,
(g) an Fc region of an immunoglobulin, which is disposed at the C-terminus of the anti-PD-L1 scFv.

In these embodiments, the Globo H/PD-L1 scFv-Fc CAR comprises in sequence, from N-terminus to C-terminus, an anti-Globo H scFv, a CD8 HTM domain, a 4-1BB co-stimulatory molecule, a cytoplasmic domain of CD3ζ, a linker, an anti-PD-L1 scFv, and an Fc region of an immunoglobulin.

Depending on intended purpose, the immunoglobulin may be IgG, IgA, IgM, IgD or IgE. According to some preferred embodiments, the immunoglobulin is IgG, for example, IgG1, IgG2, IgG3 or lgG4. In some embodiments, the constant region of the immunoglobulin contains a mutation that reduces the binding affinity of the immunoglobulin to an Fc receptor or reduces Fc effector function. For example, the constant region of the immunoglobulin may contain a mutation that eliminates the glycosylation site within the constant region of heavy chain of the immunoglobulin. In some embodiments, the constant region of the immunoglobulin contains one or more mutations, deletions, and/or insertions at an amino acid position corresponding to L234, L235, G236, G237, N297, or P331 of IgG1. In one particular embodiment, the constant region of the immunoglobulin contains a mutation at an amino acid position corresponding to N297 of IgG1. In alternative embodiments, the constant region of the immunoglobulin contains one or more mutations, deletions, and/or insertions at an amino acid position corresponding to L281, L282, G283, G284, N344, or P378 of IgG1. In certain exemplary embodiments, the immunoglobulin is IgG1, and the Globo H/PD-L1 scFv-Fc CAR comprises the amino acid sequence of SEQ ID NO: 28 or 29. According to some alternative embodiments, PD-L1 scFv-Fc is a humanized antibody; in these embodiments, the Globo H/PD-L1 scFv-Fc CAR comprises the amino acid sequence of SEQ ID NO: 30, 31, 32 or 33.

Optionally, the Fc region is linked to the anti-PD-L1 scFv via a linker, which preferably comprises the glycine (G) and/or serine (S) residues. According to some exemplary embodiments of the present disclosure, the linker for linking the Fc region to the anti-PD-L1 scFv comprises the amino acid sequence of (G₄S)₃ (SEQ ID NO: 51).

Also disclosed herein are isolated nucleic acids respectively encoding the Globo H CAR, Globo H/PD-L1 scFv CAR, and Globo H/PD-L1 scFv-Fc CAR of the present disclosure. According to certain embodiments of the present disclosure, the isolated nucleic acid encoding the Globo H CAR comprises in sequence, from 5′ end to 3′ end,
(a) a first coding sequence encoding the anti-Globo H scFv;
(b) a second coding sequence encoding the CD8 HTM domain;
(c) a third coding sequence encoding the 4-1BB molecule; and
(d) a fourth coding sequence encoding the cytoplasmic domain of CD3ζ.

According to some embodiments of the present disclosure, the first, second, third and fourth coding sequences respectively comprise the nucleotide sequences of SEQ ID NOs: 38, 39, 40 and 41. In these embodiments, the nucleic acid encoding the Globo H CAR comprises the nucleotide sequence of SEQ ID NO: 42. As could be appreciated, the present first, second, third and/or fourth coding sequences may be modified to comprise one or more degenerate nucleotides as long as the protein(s) (i.e., the anti-Globo H scFv, CD8 HTM, 4-1BB molecule and/or cytoplasmic domain of CD3ζ) encoded by the degenerate nucleotide sequence maintains the desired activity or function. The term “degenerate nucleotide sequence” (also known as “nucleotide degeneracy”) denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp). Accordingly, the nucleotide sequences comprising degenerate nucleotide(s) are intended to be included within the scope of the present disclosure, providing that the variations in the nucleotide sequence maintain at least 85% sequence identity to SEQ ID NO: 38, 39, 40, 41 or 42, such as at least 85%, 86%, 87%, 88%, 89%, 90%, 91%, 92%, 93%, 94%, 95%, 96%, 97%, 98% or 99% sequence identity to SEQ ID NO: 38, 39, 40, 41 or 42.

According to certain embodiments, the nucleic acid encoding the Globo H/PD-L1 scFv CAR comprises in sequence, from 5′ end to 3′ end,
(a) a first coding sequence encoding the anti-Globo H scFv;
(b) a second coding sequence encoding the CD8 HTM domain;
(c) a third coding sequence encoding the 4-1BB molecule;
(d) a fourth coding sequence encoding the cytoplasmic domain of CD3ζ;
(e) a linker sequence (e.g., an IRES, or a linker sequence encoding a 2A peptide); and
(f) a fifth coding sequence encoding the anti-PD-L1 scFv.

According to certain embodiments of the present disclosure, the fifth coding sequence encodes the anti-PD-L1 scFv 1G8; in these embodiments, the first, second, third, fourth and fifth coding sequences respectively comprise the nucleotide sequences of SEQ ID NOs: 38, 39, 40, 41 and 43, and the nucleic acid encoding the Globo H/PD-L1^{1G8} scFv CAR comprises the nucleotide sequence of SEQ ID NO: 45. According to certain embodiments of the present disclosure, the fifth coding sequence encodes the anti-PD-L1 scFv 3C3; in these embodiments, the first, second, third, fourth and fifth coding sequences respectively comprise the nucleotide sequences of SEQ ID NOs: 38, 39, 40, 41 and 44, and the nucleic acid encoding the Globo H/PD-L1^{1G8} scFv CAR comprises the nucleotide sequence of SEQ ID NO: 46.

As described above, the nucleic acid may be modified to comprise one or more degenerate nucleotides as long as the protein (i.e., the anti-Globo H scFv, CD8 HTM, 4-1BB molecule, cytoplasmic domain of CD3ζ and/or anti-PD-L1 scFv) encoded thereby maintains the desired activity or function. Accordingly, the nucleotide sequences comprising degenerate nucleotide(s) are intended to be included within the scope of the present disclosure, providing that the variations in the nucleotide sequence maintain at least 85% sequence identity to SEQ ID NO: 38, 39, 40, 41, 43, 44, 45 or 46, such as at least 85%, 86%, 87%, 88%, 89%, 90%, 91%, 92%, 93%, 94%, 95%, 96%, 97%, 98% or 99% sequence identity to SEQ ID NO: 38, 39, 40, 41, 43, 44, 45 or 46.

According to alternative embodiments, the nucleic acid encoding the Globo H/PD-L1 scFv-Fc CAR comprises in sequence, from 5′ end to 3′ end,
(a) a first coding sequence encoding the anti-Globo H scFv;
(b) a second coding sequence encoding the CD8 HTM domain;
(c) a third coding sequence encoding the 4-1BB molecule;
(d) a fourth coding sequence encoding the cytoplasmic domain of CD3ζ;
(e) a linker sequence (e.g., an IRES, or a linker sequence encoding a 2A peptide);
(f) a fifth coding sequence encoding the anti-PD-L1 scFv; and
(g) a sixth coding sequence encoding the Fc region of an immunoglobulin (e.g., the Fc region of IgG).

According to certain embodiments of the present disclosure, the fifth coding sequence encodes the anti-PD-L1 scFv 1G8; in these embodiments, the first, second, third, fourth, fifth and sixth coding sequences respectively comprise the nucleotide sequences of SEQ ID NOs: 38, 39, 40, 41, 43 and 47, and the nucleic acid encoding the Globo H/PD-L1^{1G8} scFv-Fc CAR comprises the nucleotide sequence of SEQ ID NO: 48. According to certain embodiments of the present disclosure, the fifth coding sequence encodes the anti-PD-L1 scFv 3C3; in these embodiments, the first, second, third, fourth, fifth and sixth coding sequences respectively comprise the nucleotide sequences of SEQ ID NOs: 38, 39, 40, 41, 44 and 47, and the nucleic acid encoding the Globo H/PD-L1^{1G8} scFv CAR comprises the nucleotide sequence of SEQ ID NO: 49.

As described above, the nucleic acid may be modified to comprise one or more degenerate nucleotides as long as the protein (i.e., the anti-Globo H scFv, CD8 HTM, 4-1BB molecule, cytoplasmic domain of CD3ζ, anti-PD-L1 scFv and/or Fc region) encoded thereby maintains the desired activity or function. Accordingly, the nucleotide sequences comprising degenerate nucleotide(s) are intended to be included within the scope of the present disclosure, providing that the variations in the nucleotide sequence maintain at least 85% sequence identity to SEQ ID NO: 38, 39, 40, 41, 43, 44, 47, 48 or 49, such as at least 85%, 86%, 87%, 88%, 89%, 90%, 91%, 92%, 93%, 94%, 95%, 96%, 97%, 98% or 99% sequence identity to SEQ ID NO: 38, 39, 40, 41, 43, 44, 47, 48 or 49.

The present invention also provides an expression vector including any of the nucleic acid as described descried above. According to some embodiments, the expression vector is an viral vector, for example, a lentiviral vector, an adenoviral vector, a retroviral vector, an adeno-associated viral vector, or a sindbis viral vector. In some exemplary embodiments, the expression vector is a lentiviral vector.

### (ii) Cells Expressing the CAR and Uses Thereof in Treating Cancers

The nucleic acid or expression vector described in Section (i) of the present disclosure is useful in producing a genetically modified cell. Specifically, the nucleic acid or expression vector may be introduced into a cell, preferably an immune cell (e.g., T cell, NK cell or macrophage), via a transfection method known in the art; for example, chemical transfection (e.g., calcium phosphate transfection, liposome transfection or non-liposome transfection), or physical transfection (e.g., microinjection, electroporation or biolistic particle delivery). Alternatively, in the case when the expression vector is a viral vector (e.g., a lentiviral vector), it may be introduced into a host cell (e.g., HEK293T cell) via a transfection method to produce the virus (e.g., lentivirus), followed by infecting the cell (e.g., T cell, NK cell or macrophage) with the virus to achieve the gene expression purpose.

The thus-produced cell (e.g., CAR-T cell) is characterized by, (a) having the CAR expressed on its cell surface that allows the cell to specifically target and destroy cancer cells; and/or (b) producing and/or secreting anti-PD-L1 antibody (i.e., anti-PD-L1 scFv or anti-PD-L1 scFv-Fc) that reduces the immunosuppression in tumor microenvironment thereby improving the anti-tumor response of the CAR-expressing cell (e.g., CAR-T cell) in solid tumors.

Accordingly, another aspect of the present disclosure pertains to a genetically modified cell (i.e., a cell expressing the CAR), and uses of the cell in the treatment of cancers.

Depending on desired purpose, the cell modified with the present nucleic acid or expression vector may be a T cell, NK cell or macrophage. According to some preferred embodiments, the genetically modified cell is a T cell (i.e., a CAR-T cell). The method of treating a cancer in a subject comprises administered to the subject an effective amount of the genetically modified cell (e.g., CAR-T cell, CAR-NK cell or CAR-macrophage) so as to alleviate or ameliorate the symptoms of the cancer.

According to certain embodiments, the subject is a mouse, in which about 1×10⁴ to 1×10⁸ (e.g., 1×10⁴, 1.5×10⁴, 2×10⁴, 2.5×10⁴, 3×10⁴, 3.5×10⁴, 4×10⁴, 4.5×10⁴, 5×10⁴, 5.5×10⁴, 6×10⁴, 6.5×10⁴, 7×10⁴, 7.5×10⁴, 8×10⁴, 8.5×10⁴, 9×10⁴, 9.5×10⁴, 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷, 9.5×10⁷, or 1×10⁸) of CAR-T cells are transferred to the subject. Preferably, about 1×10⁵ to 1×10⁷ of CAR-T cells are transferred to the subject. More preferably, about 5×10⁵ to 1×10⁶ of CAR-T cells are transferred to the mouse subject. In one specific example, about 6×10⁵ of CAR-T cells are sufficient to provide a protective and/or therapeutic effect in the mouse subject.

In general, 1×10⁶ to 1×10⁷ (e.g., 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, or 1×10⁷) CAR-T cells/Kg body weight of the subject per transplant dose are required for human CAR-T therapy. As could be appreciated, the number of CAR-T cells transferred into the human subject may vary with clinical factors, such as age, gender, underlying diseases, treatment plan, conditioning regimen and infection. A skilled artisan or medical practitioner may adjust or optimize the transferred number of CAR-T cells in accordance with desired purposes.

The genetically modified cells may be autologous to the subject (i.e., being harvested from the subject having the cancer), allogeneic to the subject (i.e., being harvested from another subject, who is of the same species as the subject having the cancer), or xenogeneic to the subject (i.e., being harvested from a donor that is of a different species relative to the subject having the cancer). Preferably, the genetically modified cells are derived from the subject being treated/administered so as to avoid transplant rejection. In the case when the genetically modified cells are allogeneic or xenogeneic to the subject, the method further comprises the step of administering to the subject an immunosuppressive treatment prior to, concurrently with, or after the administration of genetically modified cells, so as to suppress the immune response of the subject against the allogeneic or xenogeneic cells. The immunosuppression may be achieved by any agent and/or method known by a skilled artisan to prevent transplant rejection, for example, the administration of gamma irradiation or immunosuppressant.

Depending on desired purposes, the immunosuppressant may be a glucocorticoid (e.g., prednisone, budesonide, prednisolone, dexamethasone or hydrocortisone), janus kinase inhibitor (e.g., tofacitinib), calcineurin inhibitor (e.g., cyclosporine or tacrolimus), mTOR inhibitor (e.g., sirolimus or everolimus), inhibitor of inosine monophosphate dehydrogenase (IMDH inhibitor; e.g., azathioprine, leflunomide or mycophenolate), biologics or monoclonal antibody (e.g., abatacept, adalimumab, anakinra, certolizumab, etanercept, golimumab, infliximab, ixekizumab, natalizumab, rituximab, secukinumab, tocilizumab, ustekinumab, vedolizumab, basiliximab or daclizumab), or any agent known to suppress or reduce the immune response, such as methotrexate or mercaptopurine. A clinical practitioner or a skilled artisan may determine the type of immunosuppressant and treatment regimen in accordance with the physical conditions of the subject.

The genetically modified cell may be administered to the subject via any appropriate route, for example, intravenous, intraperitoneal, intraarterial or intratumoral injection. Preferably, the genetically modified cell is intravenously injected to the subject.

As would be appreciated, the present method can be applied to the subject, alone or in combination with additional therapies that have some beneficial effects on the prevention or treatment of cancers, for example, immunotherapy (e.g., the treatment of PD-1 inhibitor or PD-L1 inhibitor), surgery, chemotherapy and/or radiation therapy. According to some embodiments of the present disclosure, the genetically modified cells is administered to the subject, in combination with an PD-1 inhibitor (e.g., Nivolumab). According to some embodiments of the present disclosure, the genetically modified cells is administered to the subject, in combination with an PD-L1 inhibitor (e.g., Atezolizumab). Depending on the intended/therapeutic purpose, the present method can be applied to the subject before, during, or after the administration of the additional therapies.

Also disclosed herein is a pharmaceutical composition for the treatment of cancers. According to some embodiments of the present disclosure, the pharmaceutical kit comprises a first container containing the genetically modified cell (i.e., a CAR-T cell), and a second container containing an inhibitor of PD-L1 or an inhibitor of PD-1.

Depending on intended purpose, the PD-L1 inhibitor or PD-1 inhibitor may be Nivolumab, Atezolizumab, Avelumab, Pembrolizumab, Cemiplimab, or any other agents known to block the interaction of PD-1 and PD-L1 or inhibit the activity or function of PD-1 or PD-L1. According to one exemplary embodiment of the present disclosure, the second container contains Nivolumab. According to another exemplary embodiment of the present disclosure, the second container contains Atezolizumab.

The containers suitable for holding the genetically modified cell and PD-L1/PD-1 inhibitor may be formed from a variety of materials such as glass, or plastic. The first container may hold the genetically modified cell, in an amount effective for killing the cancer. The second container may hold the PD-L1 inhibitor or PD-1 inhibitor, in an amount effective for blocking the activity or function of PD-L1 or PD-1.

Optionally, the pharmaceutical kit may further comprise a label or package insert on or associated with the containers. The label or package insert indicates that genetically modified cell and PD-L1/PD-1 inhibitor respectively housed in the first and second containers are used for treating specified cancers.

Alternatively or additionally, the pharmaceutical kit may further comprise a third container containing a pharmaceutically acceptable buffer, such as a phosphate-buffered saline (PBS), Ringer's solution or dextrose solution. Optionally, the pharmaceutical kit may further comprises other materials desirable from a commercial and user standpoint, such as diluents, filters, needles, and syringes.

Optionally, the pharmaceutical kit further comprises a direction for the administration of the genetically modified cell and the PD-L1 inhibitor or PD-1 inhibitor.

Non-limiting examples of cancers treatable with the present method and/or pharmaceutical composition include, gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, and head and neck squamous cell carcinoma. Preferably, the cancer is a Globo H-positive cancer, i.e., the cancer expressing Globo H.

Basically, the subject treatable by the present method and/or pharmaceutical composition is a mammal, for example, human, mouse, rat, guinea pig, hamster, monkey, swine, dog, cat, horse, sheep, goat, cow, and rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Plasmid Construction

A human immunodeficiency virus (HIV)-1-based lentiviral expression vector (pLVX-EF1a-IRES) was used in this study. The DNA fragments respectively encoding Globo H scFv, CD8 hinge and transmembrane domains, co-stimulation domain 4-1BB intracellular signaling domain, and CD3ζ domain were synthesized and assembled into a CAR gene cassette. The assembled cassette was inserted into pLVX-EF1a-IRES vector via EcoRI and BamHI restriction enzyme sites. Similarly, the DNA fragments respectively encoding Globo H scFv, CD8 hinge and transmembrane domains, co-stimulation domain 4-1BB intracellular signaling domain, CD3ζ domain, T2A, signal peptide (SEQ ID NO: 50), anti-PD-L1 scFv, (G₄S)₃ (SEQ ID NO: 51) and human IgG1 Fc domain were synthesized and assembled into a CAR-PD-L1 gene cassette. The assembled cassette was inserted into pLVX-EF1a-IRES vector via EcoRI and BamHI restriction enzyme sites.

The thus-produced plasmids were respectively designated as,
(a) “Globo H CAR plasmid” (i.e., the plasmid encoding Globo H CAR);
(b) “Globo H/PD-L1^{1G8} scFv CAR plasmid” (i.e., the plasmid encoding Globo H CAR and anti-PD-L1 scFv 1G8);
(c) “Globo H/PD-L1^{3C3} scFv CAR plasmid” (i.e., the plasmid encoding Globo H CAR and anti-PD-L1 scFv 3C3);
(d) “Globo H/PD-L1^{1G8} scFv-Fc CAR plasmid” (i.e., the plasmid encoding Globo H CAR and anti-PD-L1 scFv-Fc 1G8); and
(e) “Globo H/PD-L1^{3C3} scFv-Fc CAR plasmid” (i.e., the plasmid encoding Globo H CAR and anti-PD-L1 scFv-Fc 3C3).

The plasmid encoding Globo H CAR and Atezolizumab scFv-Fc (designated as “Globo H/PD-L1^{Ate} scFv-Fc CAR plasmid”) and the plasmid encoding Globo H CAR and Nivolumab scFv-Fc (designated as “Globo H/Nivo-scFv-Fc CAR plasmid”) served as positive controls in the present study.

### Lentivirus Production

1×10⁷ 293T cells were seeded in 15 cm dish. Before transfection, culture medium was replaced by fresh DMEM medium containing 10% fetal bovine serum (FBS). Three plasmids, including pMD.G (6 ug), R8.91 (15 ug) and transfer plasmid (20 ug), were mixed with transfection reagent polycation polyethylenimine (PEI) at a volume ratio of 1:2.5, followed by incubating at room temperature for 20 minutes. Then, the mixture was added into 293T cells. 16 hours later, culture medium was changed with DMEM medium with 2% FBS. The culture supernatant containing viral particles were harvested at 48 and 72 hours post-transfection, and then mixed with concentration reagent overnight, followed by centrifuging the mixture at 1,500×g for 30 minutes at 4° C. The lentiviral particles were resuspended in media, and the viral titer was determinant by Jurkat cells infection and flow cytometer.

The thus-produced lentiviruses were respectively designated as,
(a) “Globo H CAR virus” (i.e., the lentivirus carrying the Globo H CAR plasmid);
(b) “Globo H/PD-L1^{1G8} scFv CAR virus” (i.e., the lentivirus carrying the Globo H/PD-L1^{1G8} scFv CAR plasmid);
(c) “Globo H/PD-L1^{3C3} scFv CAR virus” (i.e., the lentivirus carrying the Globo H/PD-L1^{3C3} scFv CAR plasmid);
(d) “Globo H/PD-L1^{1G8} scFv-Fc CAR virus” (i.e., the lentivirus carrying the Globo H/PD-L1^{1G8} scFv-Fc CAR plasmid); and
(e) “Globo H/PD-L1^{3C3} scFv-Fc CAR virus” (i.e., the lentivirus carrying the Globo H/PD-L1^{3C3} scFv-Fc CAR plasmid).

The lentivirus carrying the Globo H/PD-L1^{Ate} scFv-Fc CAR plasmid (designated as “Globo H/PD-L1^{Ate} scFv-Fc CAR virus”) and the lentivirus carrying the Globo H/Nivo scFv-Fc CAR plasmid (designated as “Globo H/Nivo scFv-Fc CAR virus”) served as positive controls in the present study.

### CART Cells Preparation

### (i) Isolation of Peripheral Blood Mononuclear Cells (PBMCs)

The blood sample (10 ml) isolated from healthy donor was diluted with 1× phosphate buffered saline (PBS; 10 mL) or balanced salt buffer. Lymphoprep media (15 ml) were added to the centrifuge tube, followed by carefully layering the diluted blood sample (total 20 ml) onto the Lymphoprep media solution, and then centrifuging at 800×g for 20 minutes at 15° C.-20° C. with brake off. The upper layer containing plasma and platelets was discarded using a sterile pipette. The mononuclear cell layer undisturbed at the interface was transferred to a sterile centrifuge tube (8 mL), and mixed with at least 3 volumes (about 25 ml) of 1×PBS. After centrifuging at 500×g for 10 minutes at 20° C., the supernatant was discarded, and 20 mL of 1×PBS were added to the mononuclear cells in the centrifuge tube. The tube was centrifuged at 500×g for another 10 minutes at 20° C., and then washed with 1×PBS again. The cell pellet was resuspended in media appropriate for the cell number determination.

### (ii) Isolation of Pan T Cells

After determining the cell number, the tube was centrifuged at 300×g for 10 minutes. The thus-obtained cell pellet was resuspended in buffer, and mixed with CD3 MicroBeads. The mixture was incubated for 15 minutes at 4-8° C. After washing the cells with 1-2 mL of buffer and centrifuging at 300×g for 10 minutes, the cells were resuspended in buffer and then added to the column in the magnetic field of Separator QuadroMACS™. Unlabeled cells which pass through were collected, and the column was washed with 3 mL of buffer three times. The column was removed from the separator and placed on a suitable collection tube. 5 mL of 1×PBS were added to the column, the fraction containing the magnetically labeled cells was immediately flushed out by firmly applying the plunger supplied with the column. The flowthrough was collected and the T cell number was determined.

(iii) T Cell Activation

The Dynabeads® Human T-Activator CD3/CD28 were added to the purified T cell (Beads:Cells=2:1). Culture medium were changed every 2 days.

### (iv) Viral Transduction

Primary T cells were seeded in a 6-well plate (1.8×10⁶ cells/well). Lentivirus carrying CAR-encoding nucleic acid was added to the cells (MOI=1; MOI:multiplicity of infection), followed by centrifuging at 800×g for 90 minutes, and incubating at 37° C. overnight. Then, 2 ml medium containing IL-2 (125 U/ml) were added, and the expression of GFP(Fab) was detected by flow cytometry on Day 4, Day 7 and Day 10.

### (v) CAR-T Cell Expansion

The cell density was adjusted to 5×10⁵/mL with medium. On day 4, the medium were changed, and the cells were cultured with 30 mL Bioreactor (starting with 9×10⁶ total cell for expansion; 120 rpm). On day 7, the medium were changed, and the cells were cultured with 100 mL Bioreactor (starting with 3×10⁷ total cell for expansion; 90 rpm). The CAR-T cells were harvested on day 10.

The thus-produced CAR-T cells were respectively designated as,
(a) “Globo H CAR-T cell” (i.e., the T cell transduced with the Globo H CAR virus);
(b) “Globo H/PD-L1^{1G8} scFv CAR-T cell” (i.e., the T cell transduced with the Globo H/PD-L1^{1G8} scFv CAR virus);
(c) “Globo H/PD-L1^{3C3} scFv CAR-T cell” (i.e., the T cell transduced with the Globo H/PD-L1^{3C3} scFv CAR virus);
(d) “Globo H/PD-L1^{1G8} scFv-Fc CAR-T cell” (i.e., the T cell transduced with the Globo H/PD-L1^{1G8} scFv-Fc CAR virus); and
(e) “Globo H/PD-L1^{3C3} scFv-Fc CAR-T cell” (i.e., the T cell transduced with the Globo H/PD-L1^{3C3} scFv-Fc CAR virus).

The T cell transduced with the Globo H/PD-L1^{Ate} scFv CAR virus (designated as “Globo H/PD-L1^{Ate} scFv-Fc CAR-T cell”) and the T cell transduced with the Globo H CAR/Nivo scFv-Fc CAR virus (designated as “Globo H CAR/Nivo scFv-Fc CAR-T cell”) served as positive controls in the present study.

### Cytotoxicity Assay and Cytokine Analysis

Target cells were seeded in a 96-well plate at a concentration of 2×10⁴ cells/well in triplicates. Subsequently, CAR-T cells (effector cells) were added at different effector-to target (E:T) ratios, including 1:1, 0.5:1, 0.25:1, 0.2:1 and 0.1:1 for NCI-N87 and NCI-N87/PD-L1. The cells were incubated at 37° C. for 24 hours. The supernatant was collected and subjected to enzyme-linked immunosorbent assay (ELISA) so as to determine the expression level of IFN-γ in the culture medium. The cells were washed with RPMI1640 culture medium twice, and cell counting kit (CCK-8) was used to determine the number of viable cells in the cytotoxicity assay.

### Animal Study

The NCI-N87 cells used for implantation were harvested during log phase growth and re-suspended in phosphate buffered saline (PBS) with 50% Matrigel® Basement Membrane Matrix to a concentration containing 3×10⁷ cells/mL. NCI-N87 tumor cells (3×10⁶ cells) in a dose volume of 0.1 mL were subcutaneously (SC) injected to the right front flank of mice for tumor growth. Ten days post tumor cell inoculation, when the mean tumor volume (MTV) reached approximately 117 mm³, tumor-bearing mice were randomly divided to 7 groups, in which cach group consisted of 5 mice, and administrated with CAR-T cells or vehicle solution on the same study day. Globo H CAR-T and Non-Globo H CAR-T cell suspensions (6×10⁵ CAR-T cells in a dose volume of 0.1 mL) were immediately IV injection for a single dose. Seven days after administration of CAR-T cells, anti-PD-L1 or anti-PD-1 antibody was administered twice per week (BIW) to tumor-bearing mice for 3 consecutive weeks. The day of CAR-T administration was denoted as Day 0. Tumors were measured three times per week using digimatic calipers, and the tumor volume was expressed in mm³ using the formula: TV=(w²×l)/2; where w=width and 1=length in diameter (mm) of the tumor. Tumor growth inhibition (TGI) rate was calculated using the following formula: %TGI=[1−(T/C)]×100%; where T and C represent the MTV of the treatment group and the vehicle control group, respectively.

### Example 1 Anti-Tumor Effect of GLOBO H CAR-T Cell In Vitro

The effect of GH CAR-T cells on cancer cells was examined in this example. Compared to non-GH CAR-T cells, which served as negative control group in the study, the treatment of the present Globo H CAR-T cells exhibited cytotoxic activity to cancer cells and secreted IFN-γ and Granzyme B in a dose-dependent manner (data not shown).

### Example 2 Anti-Tumor Effect of GLOBO H CAR-T Cell With or Without PD-1/PD-L1 Inhibitor

The therapeutic effect of the present Globo H CAR-T cell on cancers was evaluated in this example. As described in “Materials and Methods”, the mice bearing N87 xenografts were intravenously administered with the present Globo H CAR-T cell (6×10⁵ cells/mouse, single dose) alone, or in combination with PD-1 or PD-L1 inhibitor. The tumor volumes were measured every two or three days by using caliper, and the result was summarized in Table 1.

The data indicated that the administration of the present Globo H CAR-T cell inhibited tumor growth, and the combined treatment of the Globo H CAR-T cell and the anti-PD-1 antibody (Nivolumab) or anti-PD-L1 antibody (Atezolizumab) further improved the therapeutic efficacy (Table 1).

TABLE 1
Anti-tumor effect of specified treatment

| Treatment | Tumor growth inhibition (TGI) rate* |
|---|---|
| Globo H CAR-T | 68.4% |
| Globo H CAR-T + Nivolumab | 83.6% |
| Globo H CAR-T + Atezolizumab | 90.8% |

*The TGI rate was determined on day 28 post-treatment.

### Example 3 Expression of the Present CAR Constructs

For the purpose of evaluating the expression of the present CAR constructs, the plasmids encoding the CAR molecules were transfected into 293T cells, and the expression level of PD-L1 was determined by ELISA. According to the analytic results, each of the Globo H/PD-L1^{1G8} scFv CAR plasmid and Globo H/PD-L1^{3C3} scFv CAR plasmid was useful in expressing anti-PD-L1 scFv in 293T cells (data not shown), and each of the Globo H/PD-L1^{1G8} scFv-Fc CAR plasmid and Globo H/PD-L1^{3C3} scFv-Fc CAR plasmid was capable of expressing anti-PD-L1 scFv-Fc in 293T cells (data not shown). It was noted that the protein level of anti-PD-L1 scFv fused with Fc fragment (anti-PD-L1-scFv-Fc) expressed by the CAR Globo H/PD-L1^{3C3} scFv-Fc CAR plasmid was higher than that of anti-PD-L1 scFv expressed by the CAR Globo H/PD-L1^{3C3} scFv CAR plasmid (data no shown).

After confirming the expression of CAR-coding plasmids in mammalian cells, the lentiviruses carrying the CAR-coding plasmids were respectively transduced into primary T cells. The transduction rate was analyzed by flow cytometry as described in “Materials and Methods”. The data indicated that the transduction of Globo H CAR virus, Globo H/PD-L1^{1G8} scFv-Fc CAR virus or Globo H/PD-L1^{3C3} scFv-Fc CAR virus induced the primary T cells to express CAR (the transduction rate was about 13-30%; data not shown), in which about 0.1 to 0.15 pg/cell of anti-PD-L1 scFv-Fc were secreted by the T cells transduced with Globo H/PD-L1^{1G8} scFv-Fc CAR virus or Globo H/PD-L1^{3C3} scFv-Fc CAR virus (data not shown).

### Example 4 Anti-Tumor Effect of the Present CAR-T Cells

### 4.1 In Vitro Analysis

The cytotoxic activity of the present CAR-T cells was determined by co-incubating the CAR-T cells with cancer cells at different effector-to target (E:T) ratios as described in “Materials and Methods”.

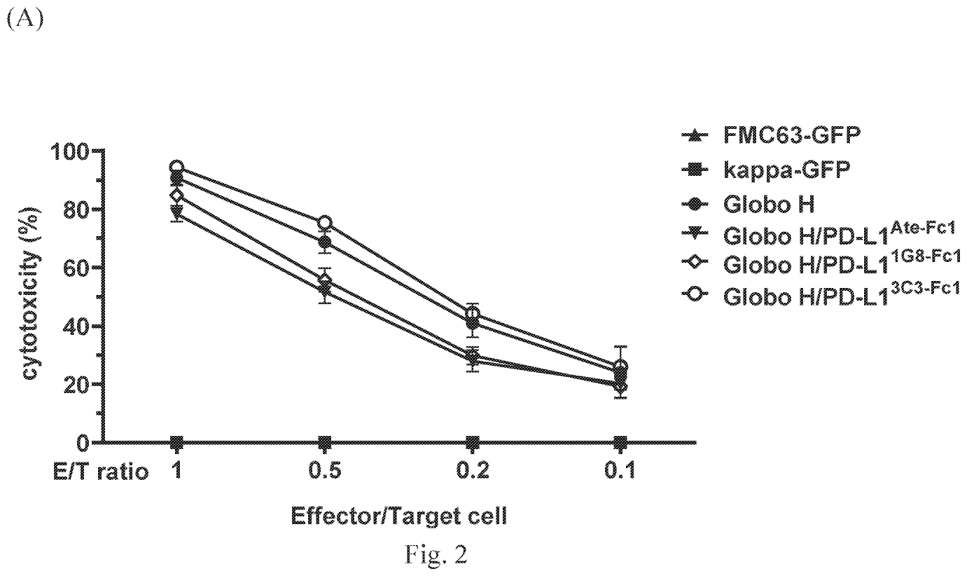

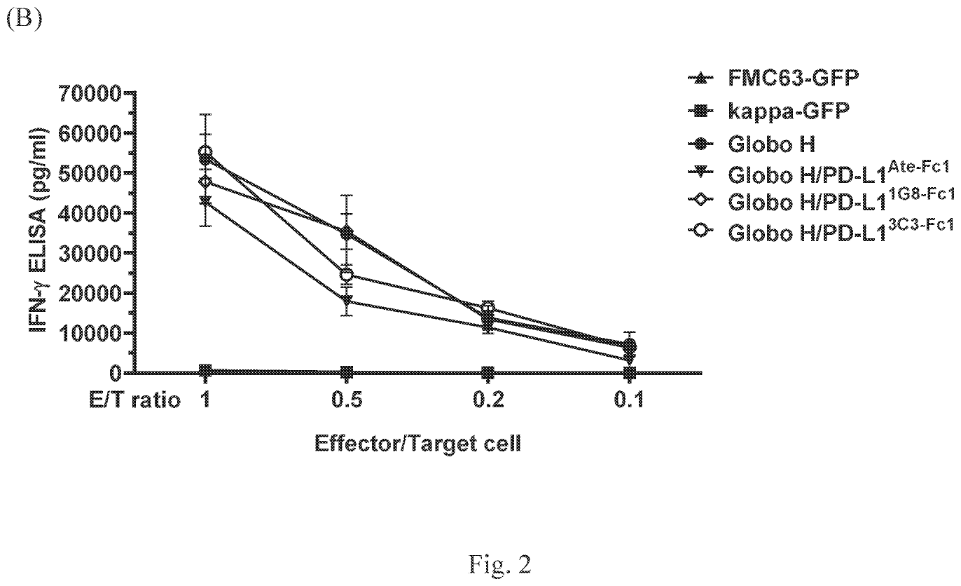

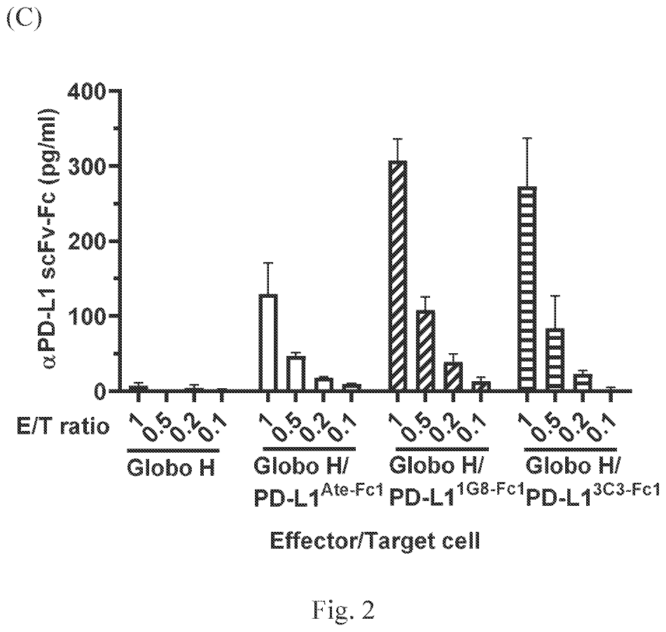

The data of FIG. 6 demonstrated that the present CAR-T cells (including the Globo H CAR-T cell, Globo H/PD-L1^{1G8} scFv-Fc CAR-T cell, and Globo H/PD-L1^{3C3} scFv-Fc CAR-T cell) exhibited cytotoxic activity to cancer cells and secreted IFN-γ in a dose-dependent manner (Panels (A) and (B) of FIG. 2). Further, the Globo H/PD-L1^{1G8} scFv-Fc CAR-T cell and Globo H/PD-L1^{3C3} scFv-Fc CAR-T cell dose-dependently secreted anti-PD-L1 scFv-Fc in culture medium (Panel (C) of FIG. 2).

### 4.2 In Vivo Analysis

The N87 gastric tumor model was used in the example to evaluate the therapeutic effect of the present CAR-T cells on cancers. As described in “Materials and Methods” of the present disclosure, single dose of CAR-T cells (6×10⁵ cells) were infused into the N87-bearing mice, and the tumor volumes were monitored every two or three days.

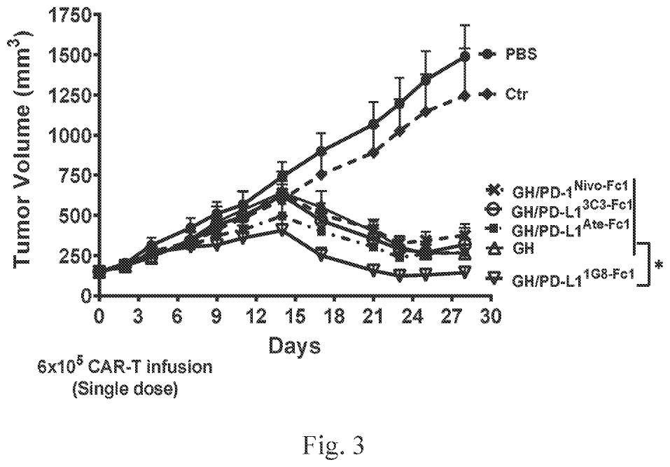

Compared to the control group, the administration of the present CAR-T cells (including the Globo H CAR-T cell, Globo H/PD-L1^{1G8} scFv-Fc CAR-T cell, and Globo H/PD-L1^{3C3} scFv-Fc CAR-T cell) significantly inhibited the tumor growth in N87 tumor model, in which the tumor growth inhibition (TGI) rate of the Globo H/PD-L1^{1G8} scFv-Fc CAR-T cell was about 90% (FIG. 3).

In conclusion, the present disclosure provides five CAR-T cells (i.e., the Globo H CAR-T cell, Globo H/PD-L1^{1G8} scFv CAR-T cell, Globo H/PD-L1^{3C3} scFv CAR-T cell, Globo H/PD-L1^{1G8} scFv-Fc CAR-T cell, and Globo H/PD-L1^{3C3} scFv-Fc CAR-T cell), each of which exhibited binding affinity and cytotoxic activity toward cancer cells. According to example of the present disclosure, the Globo H/PD-L1^{1G8} scFv CAR-T cell, Globo H/PD-L1^{3C3} scFv CAR-T cell, Globo H/PD-L1^{1G8} scFv-Fc CAR-T cell, and Globo H/PD-L1^{3C3} scFv-Fc CAR-T cell further produce and secrete anti-PD-L1 scFv-Fc that reduces the immunosuppression in tumor microenvironment thereby improving the anti-tumor response of the CAR-T cell in solid tumors.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A chimeric antigen receptor (CAR) comprising, in sequence from N-terminus to C-terminus,
(a) a first single-chain variable fragment (scFv) specific to globohexaosylceramide (Globo H);
(b) a hinge and transmembrane (HTM) domain;
(c) a co-stimulatory molecule; and
(d) a cytoplasmic domain.

2. The CAR of claim 1, wherein the HTM domain is a HTM domain of cluster of differentiation 8 (CD8); the co-stimulatory molecule is 4-1BB; and the cytoplasmic domain is a cytoplasmic domain of CD3 zeta chain (CD3ζ).

3. The CAR of claim 1, wherein
the first scFv comprises a heavy chain variable (VH) domain and a light chain variable (VL) domain, wherein the VH domain comprises a first heavy chain complementarity determining region (CDR-H1), a second heavy chain CDR (CDR-H2) and a third heavy chain CDR (CDR-H3), and the VL domain comprises a first light chain CDR (CDR-L1), a second light chain CDR (CDR-L2) and a third light chain CDR (CDR-L3), wherein the CDR-H1, CDR-H2 and CDR-H3 respectively comprise the amino acid sequences of SEQ ID NOs: 1-3, and the CDR-L1, CDR-L2 and CDR-L3 respectively comprise the amino acid sequences of SEQ ID NOs: 4-6;
the HTM domain of CD8 comprises the amino acid sequence of SEQ ID NO: 9;
the 4-1BB co-stimulatory molecule comprises the amino acid sequence of SEQ ID NO: 10; and
the cytoplasmic domain of CD3ζ comprises the amino acid sequence of SEQ ID NO: 11.

4. The CAR of claim 3, wherein the VH and VL domains respectively comprise the amino acid sequences of SEQ ID NOs: 7 and 8.

5. The CAR of claim 1, further comprising,
(e) a second scFv specific to programmed death-ligand 1 (PD-L1); and
(f) a linker linking the second scFv to the C-terminus of the cytoplasmic domain, wherein the linker is a 2A peptide or an internal ribosomal entry site (IRES).

6. The CAR of claim 5, wherein the second scFv comprises a VH domain and a VL domain, wherein the VH domain comprises a CDR-H1, a CDR-H2 and a CDR-H3, and the VL domain comprises a CDR-L1, a CDR-L2 and a CDR-L3, wherein
the CDR-H1, CDR-H2 and CDR-H3 respectively comprise the amino acid sequences of SEQ ID NOs: 12-14, and the CDR-L1, CDR-L2 and CDR-L3 respectively comprise the amino acid sequences of SEQ ID NOs: 15-17; or
the CDR-H1, CDR-H2 and CDR-H3 respectively comprise the amino acid sequences of SEQ ID NOs: 20-22, and the CDR-L1, CDR-L2 and CDR-L3 respectively comprise the amino acid sequences of SEQ ID NOs: 23-25.

7. The CAR of claim 6, wherein
the VH domain and VL domain of the second scFv respectively comprise the amino acid sequences of SEQ ID NOs: 18 and 19; or
the VH domain and VL domains of the second scFv respectively comprise the amino acid sequences of SEQ ID NOs: 26 and 27.

8. The CAR of claim 5, further comprising,
(g) a fragment crystallizable region (Fc region) of an immunoglobulin that is disposed at the C-terminus of the second scFv.

9. The CAR of claim 8, wherein the immunoglobulin is immunoglobulin G (IgG).

10. The CAR of claim 9, wherein the CAR comprises the amino acid sequence of SEQ ID NO: 28 or 29.

11. An isolated nucleic acid encoding the chimeric antigen receptor (CAR) of claim 1, comprising, in sequence from 5′ end to 3′ end, a first, a second, a third and a fourth coding sequences respectively encoding the first scFv, HTM domain, co-stimulatory molecule and cytoplasmic domain of the CAR, wherein the first, second, third and fourth coding sequences respectively comprise the nucleotide sequences at least 85% identical to SEQ ID NOs: 38, 39, 40 and 41.

12. The isolated nucleic acid of claim 11, further comprising a linker sequence and a fifth coding sequence, wherein the fifth coding sequence is linked to the 3′ end of the fourth coding sequence via the linker sequence, wherein
the linker sequence is an IRES or encodes a 2A peptide;
the fifth coding sequence encodes a second scFv specific to PD-L1, and comprises a nucleotide sequence at least 85% identical to SEQ ID NO: 43 or 44.

13. The isolated nucleic acid of claim 12, further comprising a sixth coding sequence linked to the 3′ end of the fifth coding sequence, wherein the sixth coding sequence encodes a Fc region of an immunoglobulin, and comprises a nucleotide sequence at least 85% identical to SEQ ID NO: 47.

14. A method of treating a cancer in a subject, comprising administering to the subject an effective amount of an isolated immune cell expressing the CAR of claim 1, so as to alleviate or ameliorate the symptoms of the cancer.

15. The method of claim 14, wherein the cancer is gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma.

16. The method of claim 14, wherein the cancer is a Globo H-expressing cancer.

17. The method of claim 14, wherein the isolated immune cell is a T cell, a natural killer (NK) cell, or a macrophage.

18. A pharmaceutical kit comprising an isolated immune cell expressing the CAR of claim 1, and an inhibitor of PD-L1 or an inhibitor of programmed death 1 (PD-1).

19. The pharmaceutical kit of claim 18, wherein the isolated immune cell is a T cell, a natural killer (NK) cell, or a macrophage.

20. The pharmaceutical kit of claim 18, wherein the inhibitor of PD-L1 is Atezolizumab, and the inhibitor of programmed death 1 (PD-1) is Nivolumab.


SEQUENCE LISTING (51 sequences)

SEQ ID NO: 1 - protein, 10 aa, synthetic construct
GYISSDQILN

SEQ ID NO: 2 - protein, 17 aa, synthetic construct
RIYPVTGVTQYNHKFVG

SEQ ID NO: 3 - protein, 6 aa, synthetic construct
GETFDS

SEQ ID NO: 4 - protein, 17 aa, synthetic construct
KSNQNLLWSGNRRYTLV

SEQ ID NO: 5 - protein, 7 aa, synthetic construct
WASDRSF

SEQ ID NO: 6 - protein, 9 aa, synthetic construct
QQHLDIPYT

SEQ ID NO: 7 - protein, 115 aa, synthetic construct
EIQLVQSGGGLAQPGGSIRLSCAPSGYISSDQILNWVKKAPGKCLEWIGRIYPVTGVTQYNHKFVGKATFSVDRSKDTVYMQMNSLRAEDTGVYYCGRGETFDSWGQGTLLTVSS

SEQ ID NO: 8 - protein, 114 aa, synthetic construct
DIQLTQSISSLSVSVGDRVTINCKSNQNLLWSGNRRYTLVWHQWKPGKSPKPLITWASDRSFGVPSRFSGSGSVTDFTLTISSVQPEDFAVYFCQQHLDIPYTFGCGTKLEIKR

SEQ ID NO: 9 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 10 - protein, 42 aa, synthetic construct
KRGRKKLLYIFKQPFMRPVQTTQEEDGCSCRFPEEEEGGCEL

SEQ ID NO: 11 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 10 aa, synthetic construct
GYSITSDYWN

SEQ ID NO: 13 - protein, 16 aa, synthetic construct
YISYTGSTYYNPSLKS

SEQ ID NO: 14 - protein, 10 aa, synthetic construct
RGEWLSPFAY

SEQ ID NO: 15 - protein, 17 aa, synthetic construct
KSSQSLLYSSNQKNSLA

SEQ ID NO: 16 - protein, 7 aa, synthetic construct
WASTRES

SEQ ID NO: 17 - protein, 9 aa, synthetic construct
QQYYTYPFT

SEQ ID NO: 18 - protein, 118 aa, synthetic construct
QVQLQESGPGLAKPSQTLSLTCSVTGYSITSDYWNWIRKFPGNKLEFMGYISYTGSTYYNPSLKSRISITRDTSKNQYYLQLNSVTTEDTATYYCARRGEWLSPFAYWGQGTLVTVSA

SEQ ID NO: 19 - protein, 113 aa, synthetic construct
DIVMSQSPSSLGVSVGEKITMSCKSSQSLLYSSNQKNSLAWYQQKPGQSPKLLIYWASTRESGVPDRFTGSGSGTDFTLTISSVKSEDLAVYYCQQYYTYPFTFGAGTNLELK

SEQ ID NO: 20 - protein, 10 aa, synthetic construct
GYSITSDYWD

SEQ ID NO: 21 - protein, 16 aa, synthetic construct
YISYTGSTYYNPSLRS

SEQ ID NO: 22 - protein, 10 aa, synthetic construct
RGGWLSPFVY

SEQ ID NO: 23 - protein, 17 aa, synthetic construct
KSRQSLLFSSNQKNSLA

SEQ ID NO: 24 - protein, 7 aa, synthetic construct
WASTRES

SEQ ID NO: 25 - protein, 9 aa, synthetic construct
QQYYTYPFT

SEQ ID NO: 26 - protein, 118 aa, synthetic construct
EVQLQESGPGLTKPSQTLSLTCSVTGYSITSDYWDWIRKFPGNKLEYMGYISYTGSTYYNPSLRSRISITRDTSKNQYYLQLSSVTSEDSATYYCARRGGWLSPFVYWGQGTLVTVSA

SEQ ID NO: 27 - protein, 113 aa, synthetic construct
DTVMSQSPSSLGVSVGERVTLTCKSRQSLLFSSNQKNSLAWYQQKPGQSPKLLIYWASTRESGVPDRFTGSGSGTDFTLTISSVKSEDLAVYYCQQYYTYPFTFGAGTSLELK

SEQ ID NO: 28 - protein, 993 aa, synthetic construct
EIQLVQSGGGLAQPGGSIRLSCAPSGYISSDQILNWVKKAPGKCLEWIGRIYPVTGVTQYNHKFVGKATFSVDRSKDTVYMQMNSLRAEDTGVYYCGRGETFDSWGQGTLLTVSSGGGGSGGGGSGGGGSDIQLTQSISSLSVSVGDRVTINCKSNQNLLWSGNRRYTLVWHQWKPGKSPKPLITWASDRSFGVPSRFSGSGSVTDFTLTISSVQPEDFAVYFCQQHLDIPYTFGCGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRGSGEGRGSLLTCGDVEENPGPMDFQVQIISFLLISASVIMSRGGQVQLQESGPGLAKPSQTLSLTCSVTGYSITSDYWNWIRKFPGNKLEFMGYISYTGSTYYNPSLKSRISITRDTSKNQYYLQLNSVTTEDTATYYCARRGEWLSPFAYWGQGTLVTVSAGGGGSGGGGSGGGGSDIVMSQSPSSLGVSVGEKITMSCKSSQSLLYSSNQKNSLAWYQQKPGQSPKLLIYWASTRESGVPDRFTGSGSGTDFTLTISSVKSEDLAVYYCQQYYTYPFTFGAGTNLELKLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 993 aa, synthetic construct
EIQLVQSGGGLAQPGGSIRLSCAPSGYISSDQILNWVKKAPGKCLEWIGRIYPVTGVTQYNHKFVGKATFSVDRSKDTVYMQMNSLRAEDTGVYYCGRGETFDSWGQGTLLTVSSGGGGSGGGGSGGGGSDIQLTQSISSLSVSVGDRVTINCKSNQNLLWSGNRRYTLVWHQWKPGKSPKPLITWASDRSFGVPSRFSGSGSVTDFTLTISSVQPEDFAVYFCQQHLDIPYTFGCGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRGSGEGRGSLLTCGDVEENPGPMDFQVQIISFLLISASVIMSRGGEVQLQESGPGLTKPSQTLSLTCSVTGYSITSDYWDWIRKFPGNKLEYMGYISYTGSTYYNPSLRSRISITRDTSKNQYYLQLSSVTSEDSATYYCARRGGWLSPFVYWGQGTLVTVSAGGGGSGGGGSGGGGSDTVMSQSPSSLGVSVGERVTLTCKSRQSLLFSSNQKNSLAWYQQKPGQSPKLLIYWASTRESGVPDRFTGSGSGTDFTLTISSVKSEDLAVYYCQQYYTYPFTFGAGTSLELKLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 992 aa, synthetic construct
EIQLVQSGGGLAQPGGSIRLSCAPSGYISSDQILNWVKKAPGKCLEWIGRIYPVTGVTQYNHKFVGKATFSVDRSKDTVYMQMNSLRAEDTGVYYCGRGETFDSWGQGTLLTVSSGGGGSGGGGSGGGGSDIQLTQSISSLSVSVGDRVTINCKSNQNLLWSGNRRYTLVWHQWKPGKSPKPLITWASDRSFGVPSRFSGSGSVTDFTLTISSVQPEDFAVYFCQQHLDIPYTFGCGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRGSGEGRGSLLTCGDVEENPGPMDFQVQIISFLLISASVIMSRGEVQLQESGPGLVKPSQTLSLTCTVSGYSITSDYWNWIRKPPGKGLEYMGYISYTGSTYYNPSLKSRITISRDTSKNQYSLKLSSVTAADTAVYYCARRGEWLSPFAYWGQGTLVTVSSGGGGSGGGGSGGGGSDIQMTQSPSSLSASVGDRVTITCKSSQSLLYSSNQKNSLAWYQQKPGKAPKLLIYWASTRESGVPSRFSGSGSGTDFTLTISSLQPEDFATYYCQQYYTYPFTFGQGTKLEIKLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 992 aa, synthetic construct
EIQLVQSGGGLAQPGGSIRLSCAPSGYISSDQILNWVKKAPGKCLEWIGRIYPVTGVTQYNHKFVGKATFSVDRSKDTVYMQMNSLRAEDTGVYYCGRGETFDSWGQGTLLTVSSGGGGSGGGGSGGGGSDIQLTQSISSLSVSVGDRVTINCKSNQNLLWSGNRRYTLVWHQWKPGKSPKPLITWASDRSFGVPSRFSGSGSVTDFTLTISSVQPEDFAVYFCQQHLDIPYTFGCGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRGSGEGRGSLLTCGDVEENPGPMDFQVQIISFLLISASVIMSRGEVQLQESGPGLVKPSQTLSLTCTVSGYSITSDYWNWIRKPPGKGLEYMGYISYTGSTYYNPSLKSRITISRDTSKNQYSLKLSSVTAADTAVYYCARRGEWLSPFAYWGQGTLVTVSSGGGGSGGGGSGGGGSDIVMTQSPDSLAVSLGERATINCKSSQSLLYSSNQKNSLAWYQQKPGQPPKLLIYWASTRESGVPDRFSGSGSGTDFTLTISSLQAEDVAVYYCQQYYTYPFTFGQGTKLEIKLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 992 aa, synthetic construct
EIQLVQSGGGLAQPGGSIRLSCAPSGYISSDQILNWVKKAPGKCLEWIGRIYPVTGVTQYNHKFVGKATFSVDRSKDTVYMQMNSLRAEDTGVYYCGRGETFDSWGQGTLLTVSSGGGGSGGGGSGGGGSDIQLTQSISSLSVSVGDRVTINCKSNQNLLWSGNRRYTLVWHQWKPGKSPKPLITWASDRSFGVPSRFSGSGSVTDFTLTISSVQPEDFAVYFCQQHLDIPYTFGCGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRGSGEGRGSLLTCGDVEENPGPMDFQVQIISFLLISASVIMSRGEVQLQESGPGLVKPSQTLSLTCTVSGYSITSDYWDWIRKPPGKGLEYMGYISYTGSTYYNPSLRSRITISRDTSKNQYSLKLSSVTAADTAVYYCARRGGWLSPFVYWGQGTLVTVSSGGGGSGGGGSGGGGSDIQMTQSPSSLSASVGDRVTITCKSRQSLLFSSNQKNSLAWYQQKPGKAPKLLIYWASTRESGVPSRFSGSGSGTDFTLTISSLQPEDFATYYCQQYYTYPFTFGQGTKLEIKLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 992 aa, synthetic construct
EIQLVQSGGGLAQPGGSIRLSCAPSGYISSDQILNWVKKAPGKCLEWIGRIYPVTGVTQYNHKFVGKATFSVDRSKDTVYMQMNSLRAEDTGVYYCGRGETFDSWGQGTLLTVSSGGGGSGGGGSGGGGSDIQLTQSISSLSVSVGDRVTINCKSNQNLLWSGNRRYTLVWHQWKPGKSPKPLITWASDRSFGVPSRFSGSGSVTDFTLTISSVQPEDFAVYFCQQHLDIPYTFGCGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRGSGEGRGSLLTCGDVEENPGPMDFQVQIISFLLISASVIMSRGEVQLQESGPGLVKPSQTLSLTCTVSGYSITSDYWDWIRKPPGKGLEYMGYISYTGSTYYNPSLRSRITISRDTSKNQYSLKLSSVTAADTAVYYCARRGGWLSPFVYWGQGTLVTVSSGGGGSGGGGSGGGGSDIVMTQSPDSLAVSLGERATINCKSRQSLLFSSNQKNSLAWYQQKPGQPPKLLIYWASTRESGVPDRFSGSGSGTDFTLTISSLQAEDVAVYYCQQYYTYPFTFGQGTKLEIKLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 18 aa, synthetic construct
EGRGSLLTCGDVEENPGP

SEQ ID NO: 35 - protein, 19 aa, synthetic construct
ATNFSLLKQAGDVEENPGP

SEQ ID NO: 36 - protein, 20 aa, synthetic construct
QCTNYALLKLAGDVESNPGP

SEQ ID NO: 37 - protein, 22 aa, synthetic construct
VKQTLNFDLLKLAGDVESNPGP

SEQ ID NO: 38 - DNA, 732 bp, synthetic construct
  source 1..732: mol_type = other DNA
gagatccagctggtgcagtctggcggaggactggctcagcctggcggctctatcagactgagctgtgcccccagcggctacatcagcagcgaccagatcctgaactgggtcaagaaggcccctggcaagtgcctggaatggatcggcagaatctaccccgtgaccggcgtgacccagtacaaccacaagttcgtgggcaaggccaccttcagcgtggacagatccaaggacaccgtgtacatgcagatgaacagcctgagagccgaggacaccggcgtgtactactgcggcagaggcgagacattcgacagctggggccagggcacactgctgaccgtgtcatctggtggaggcggtagcggcggaggaggatccggcggtggcggaagcgacatccagctgacccagagcatcagcagcctgagcgtgtccgtgggcgacagagtgaccatcaactgcaagagcaaccagaacctgctgtggagcggcaacagacggtacaccctcgtgtggcaccagtggaagcctggcaagagccccaagcccctgatcacatgggccagcgacagatccttcggcgtgcccagcagatttagcggcagcggctctgtgaccgacttcaccctgaccatcagctccgtgcagcccgaggacttcgccgtgtacttctgccagcagcacctggacatcccttacaccttcggctgcggcaccaagctggaaatcaaacgt

SEQ ID NO: 39 - DNA, 207 bp, synthetic construct
  source 1..207: mol_type = other DNA
acgacaaccccagctcctaggccaccaacaccagctccaaccatcgcttcccagccactgtctctgaggccagaggcttgtagacctgctgctggaggagctgtgcacaccaggggactggactttgcctgcgatatctatatctgggctccactggctggaacatgtggcgtgctgctgctgagcctggtcatcaccctgtattgc

SEQ ID NO: 40 - DNA, 126 bp, synthetic construct
  source 1..126: mol_type = other DNA
aagaggggcagaaagaagctgctgtacatcttcaagcagccttttatgaggccagtgcagacaacccaggaggaggacggctgctcctgtagattcccagaggaggaggagggaggatgtgagctg

SEQ ID NO: 41 - DNA, 336 bp, synthetic construct
  source 1..336: mol_type = other DNA
agggtgaagttttctcggagcgccgatgctcccgcctatcagcagggccagaaccagctgtacaacgagctgaatctgggccggagggaggagtacgacgtgctggataagagaaggggacgggaccccgagatgggaggcaagccacggaggaagaacccccaggagggcctgtataatgagctgcagaaggacaagatggctgaggcctactccgagatcggcatgaagggagagagacgccggggcaagggacacgatggcctgtatcagggcctgtctacagccaccaaggacacctacgatgctctgcacatgcaggccctgcctccaaga

SEQ ID NO: 42 - DNA, 1401 bp, synthetic construct
  source 1..1401: mol_type = other DNA
gagatccagctggtgcagtctggcggaggactggctcagcctggcggctctatcagactgagctgtgcccccagcggctacatcagcagcgaccagatcctgaactgggtcaagaaggcccctggcaagtgcctggaatggatcggcagaatctaccccgtgaccggcgtgacccagtacaaccacaagttcgtgggcaaggccaccttcagcgtggacagatccaaggacaccgtgtacatgcagatgaacagcctgagagccgaggacaccggcgtgtactactgcggcagaggcgagacattcgacagctggggccagggcacactgctgaccgtgtcatctggtggaggcggtagcggcggaggaggatccggcggtggcggaagcgacatccagctgacccagagcatcagcagcctgagcgtgtccgtgggcgacagagtgaccatcaactgcaagagcaaccagaacctgctgtggagcggcaacagacggtacaccctcgtgtggcaccagtggaagcctggcaagagccccaagcccctgatcacatgggccagcgacagatccttcggcgtgcccagcagatttagcggcagcggctctgtgaccgacttcaccctgaccatcagctccgtgcagcccgaggacttcgccgtgtacttctgccagcagcacctggacatcccttacaccttcggctgcggcaccaagctggaaatcaaacgtacgacaaccccagctcctaggccaccaacaccagctccaaccatcgcttcccagccactgtctctgaggccagaggcttgtagacctgctgctggaggagctgtgcacaccaggggactggactttgcctgcgatatctatatctgggctccactggctggaacatgtggcgtgctgctgctgagcctggtcatcaccctgtattgcaagaggggcagaaagaagctgctgtacatcttcaagcagccttttatgaggccagtgcagacaacccaggaggaggacggctgctcctgtagattcccagaggaggaggagggaggatgtgagctgagggtgaagttttctcggagcgccgatgctcccgcctatcagcagggccagaaccagctgtacaacgagctgaatctgggccggagggaggagtacgacgtgctggataagagaaggggacgggaccccgagatgggaggcaagccacggaggaagaacccccaggagggcctgtataatgagctgcagaaggacaagatggctgaggcctactccgagatcggcatgaagggagagagacgccggggcaagggacacgatggcctgtatcagggcctgtctacagccaccaaggacacctacgatgctctgcacatgcaggccctgcctccaaga

SEQ ID NO: 43 - DNA, 738 bp, synthetic construct
  source 1..738: mol_type = other DNA
caggtccagctgcaagagtctggccctggactggccaagcctagccaaacactgagcctgacctgtagcgtgaccggctacagcatcaccagcgactactggaactggatccggaagttccccggcaacaagctggaattcatgggctacatcagctacaccggcagcacctactacaaccccagcctgaagtcccggatctccatcaccagagacaccagcaagaaccagtactacctgcagctgaacagcgtgaccaccgaggataccgccacctactattgcgctagaagaggcgagtggctgagcccctttgcctattggggacagggcacactggtcacagtttctgctggtggcggaggatctggcggaggtggaagcggcggaggcggatctgatatcgtgatgagccagtctcctagcagcctgggagtgtctgtgggcgagaagatcaccatgagctgcaagtccagccagagcctgctgtactccagcaaccagaagaacagcctggcctggtatcagcagaagcccggacagtctcccaagctgctgatctactgggccagcaccagagaaagcggcgtgcccgatagattcacaggcagcggcagcggaaccgacttcaccctgacaatcagcagcgtgaagtccgaggacctggccgtgtactactgccagcagtactacacataccccttcaccttcggagccggcaccaacctggaactgaag

SEQ ID NO: 44 - DNA, 738 bp, synthetic construct
  source 1..738: mol_type = other DNA
gaggtccagctgcaagagtctggccctggactgaccaagcctagccagacactgagcctgacctgtagcgtgaccggctacagcatcaccagcgactactgggactggatcagaaagttccccggcaacaagctcgagtacatgggctacatcagctacaccggcagcacctactacaaccccagcctgagaagccggatctccatcaccagagacaccagcaagaaccagtactacctgcagctgagcagcgtgaccagcgaggatagcgccacctactattgcgctagaagaggcggctggctgagcccctttgtgtattggggacagggcaccctggtcacagtttctgctggtggcggaggatctggcggaggtggaagcggcggaggcggatctgatacagtgatgagccagtctcctagcagcctgggagtgtctgtgggcgagagagtgacactgacctgcaagagcagacagagcctgctgttcagcagcaaccagaagaacagcctggcctggtatcagcagaagcccggacagtctcccaagctgctgatctactgggccagcaccagagaaagcggcgtgcccgatagattcacaggctctggcagcggcaccgacttcaccctgacaatcagctccgtgaagtccgaggacctggccgtgtactactgccagcagtactacacataccccttcaccttcggagccggcaccagcctggaactgaaa

SEQ ID NO: 45 - DNA, 2280 bp, synthetic construct
  source 1..2280: mol_type = other DNA
gagatccagctggtgcagtctggcggaggactggctcagcctggcggctctatcagactgagctgtgcccccagcggctacatcagcagcgaccagatcctgaactgggtcaagaaggcccctggcaagtgcctggaatggatcggcagaatctaccccgtgaccggcgtgacccagtacaaccacaagttcgtgggcaaggccaccttcagcgtggacagatccaaggacaccgtgtacatgcagatgaacagcctgagagccgaggacaccggcgtgtactactgcggcagaggcgagacattcgacagctggggccagggcacactgctgaccgtgtcatctggtggaggcggtagcggcggaggaggatccggcggtggcggaagcgacatccagctgacccagagcatcagcagcctgagcgtgtccgtgggcgacagagtgaccatcaactgcaagagcaaccagaacctgctgtggagcggcaacagacggtacaccctcgtgtggcaccagtggaagcctggcaagagccccaagcccctgatcacatgggccagcgacagatccttcggcgtgcccagcagatttagcggcagcggctctgtgaccgacttcaccctgaccatcagctccgtgcagcccgaggacttcgccgtgtacttctgccagcagcacctggacatcccttacaccttcggctgcggcaccaagctggaaatcaagagaaccaccaccccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctaagcggggaagcggagagggcagaggatccctgctcacctgcggcgacgtggaggagaatcctggacctatggattttcaggtgcagattatcagcttcctgctaatcagtgcttcagtcataatgtctagagggggacaggtccagctgcaagagtctggccctggactggccaagcctagccaaacactgagcctgacctgtagcgtgaccggctacagcatcaccagcgactactggaactggatccggaagttccccggcaacaagctggaattcatgggctacatcagctacaccggcagcacctactacaaccccagcctgaagtcccggatctccatcaccagagacaccagcaagaaccagtactacctgcagctgaacagcgtgaccaccgaggataccgccacctactattgcgctagaagaggcgagtggctgagcccctttgcctattggggacagggcacactggtcacagtttctgctggtggcggaggatctggcggaggtggaagcggcggaggcggatctgatatcgtgatgagccagtctcctagcagcctgggagtgtctgtgggcgagaagatcaccatgagctgcaagtccagccagagcctgctgtactccagcaaccagaagaacagcctggcctggtatcagcagaagcccggacagtctcccaagctgctgatctactgggccagcaccagagaaagcggcgtgcccgatagattcacaggcagcggcagcggaaccgacttcaccctgacaatcagcagcgtgaagtccgaggacctggccgtgtactactgccagcagtactacacataccccttcaccttcggagccggcaccaacctggaactgaag

SEQ ID NO: 46 - DNA, 2280 bp, synthetic construct
  source 1..2280: mol_type = other DNA
gagatccagctggtgcagtctggcggaggactggctcagcctggcggctctatcagactgagctgtgcccccagcggctacatcagcagcgaccagatcctgaactgggtcaagaaggcccctggcaagtgcctggaatggatcggcagaatctaccccgtgaccggcgtgacccagtacaaccacaagttcgtgggcaaggccaccttcagcgtggacagatccaaggacaccgtgtacatgcagatgaacagcctgagagccgaggacaccggcgtgtactactgcggcagaggcgagacattcgacagctggggccagggcacactgctgaccgtgtcatctggtggaggcggtagcggcggaggaggatccggcggtggcggaagcgacatccagctgacccagagcatcagcagcctgagcgtgtccgtgggcgacagagtgaccatcaactgcaagagcaaccagaacctgctgtggagcggcaacagacggtacaccctcgtgtggcaccagtggaagcctggcaagagccccaagcccctgatcacatgggccagcgacagatccttcggcgtgcccagcagatttagcggcagcggctctgtgaccgacttcaccctgaccatcagctccgtgcagcccgaggacttcgccgtgtacttctgccagcagcacctggacatcccttacaccttcggctgcggcaccaagctggaaatcaagagaaccaccaccccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctaagcggggaagcggagagggcagaggatccctgctcacctgcggcgacgtggaggagaatcctggacctatggattttcaggtgcagattatcagcttcctgctaatcagtgcttcagtcataatgtctagagggggcgaggtccagctgcaagagtctggccctggactgaccaagcctagccagacactgagcctgacctgtagcgtgaccggctacagcatcaccagcgactactgggactggatcagaaagttccccggcaacaagctcgagtacatgggctacatcagctacaccggcagcacctactacaaccccagcctgagaagccggatctccatcaccagagacaccagcaagaaccagtactacctgcagctgagcagcgtgaccagcgaggatagcgccacctactattgcgctagaagaggcggctggctgagcccctttgtgtattggggacagggcaccctggtcacagtttctgctggtggcggaggatctggcggaggtggaagcggcggaggcggatctgatacagtgatgagccagtctcctagcagcctgggagtgtctgtgggcgagagagtgacactgacctgcaagagcagacagagcctgctgttcagcagcaaccagaagaacagcctggcctggtatcagcagaagcccggacagtctcccaagctgctgatctactgggccagcaccagagaaagcggcgtgcccgatagattcacaggctctggcagcggcaccgacttcaccctgacaatcagctccgtgaagtccgaggacctggccgtgtactactgccagcagtactacacataccccttcaccttcggagccggcaccagcctggaactgaaa

SEQ ID NO: 47 - DNA, 681 bp, synthetic construct
  source 1..681: mol_type = other DNA
gacaagacccacacctgtcctccatgccctgcccctgagctgctgggcggaccctccgtgttcctgttccctccaaagcctaaggacaccctgatgatctcccggacccctgaagtgacctgcgtggtggtggacgtgtcccacgaggaccctgaagtgaagttcaattggtacgtggacggcgtggaagtgcacaacgccaagaccaagcctagagaggaacagtacgccagcacctacagagtggtgtccgtgctgacagtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctggtcaagggcttctacccttccgatatcgccgtggaatgggagagcaatggccagccagagaacaactacaagacaacccctcctgtgctggacagcgacggctcattcttcctgtacagcaagctgaccgtggacaagtccagatggcagcagggcaacgtgttcagctgcagcgtgatgcacgaggccctgcacaaccactacacccagaagtccctgtctctgagccccggcaaa

SEQ ID NO: 48 - DNA, 2979 bp, synthetic construct
  source 1..2979: mol_type = other DNA
gagatccagctggtgcagtctggcggaggactggctcagcctggcggctctatcagactgagctgtgcccccagcggctacatcagcagcgaccagatcctgaactgggtcaagaaggcccctggcaagtgcctggaatggatcggcagaatctaccccgtgaccggcgtgacccagtacaaccacaagttcgtgggcaaggccaccttcagcgtggacagatccaaggacaccgtgtacatgcagatgaacagcctgagagccgaggacaccggcgtgtactactgcggcagaggcgagacattcgacagctggggccagggcacactgctgaccgtgtcatctggtggaggcggtagcggcggaggaggatccggcggtggcggaagcgacatccagctgacccagagcatcagcagcctgagcgtgtccgtgggcgacagagtgaccatcaactgcaagagcaaccagaacctgctgtggagcggcaacagacggtacaccctcgtgtggcaccagtggaagcctggcaagagccccaagcccctgatcacatgggccagcgacagatccttcggcgtgcccagcagatttagcggcagcggctctgtgaccgacttcaccctgaccatcagctccgtgcagcccgaggacttcgccgtgtacttctgccagcagcacctggacatcccttacaccttcggctgcggcaccaagctggaaatcaagagaaccaccaccccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctaagcggggaagcggagagggcagaggatccctgctcacctgcggcgacgtggaggagaatcctggacctatggattttcaggtgcagattatcagcttcctgctaatcagtgcttcagtcataatgtctagagggggacaggtccagctgcaagagtctggccctggactggccaagcctagccaaacactgagcctgacctgtagcgtgaccggctacagcatcaccagcgactactggaactggatccggaagttccccggcaacaagctggaattcatgggctacatcagctacaccggcagcacctactacaaccccagcctgaagtcccggatctccatcaccagagacaccagcaagaaccagtactacctgcagctgaacagcgtgaccaccgaggataccgccacctactattgcgctagaagaggcgagtggctgagcccctttgcctattggggacagggcacactggtcacagtttctgctggtggcggaggatctggcggaggtggaagcggcggaggcggatctgatatcgtgatgagccagtctcctagcagcctgggagtgtctgtgggcgagaagatcaccatgagctgcaagtccagccagagcctgctgtactccagcaaccagaagaacagcctggcctggtatcagcagaagcccggacagtctcccaagctgctgatctactgggccagcaccagagaaagcggcgtgcccgatagattcacaggcagcggcagcggaaccgacttcaccctgacaatcagcagcgtgaagtccgaggacctggccgtgtactactgccagcagtactacacataccccttcaccttcggagccggcaccaacctggaactgaagcttggcggaggaggcagcgacaagacccacacctgtcctccatgccctgcccctgagctgctgggcggaccctccgtgttcctgttccctccaaagcctaaggacaccctgatgatctcccggacccctgaagtgacctgcgtggtggtggacgtgtcccacgaggaccctgaagtgaagttcaattggtacgtggacggcgtggaagtgcacaacgccaagaccaagcctagagaggaacagtacgccagcacctacagagtggtgtccgtgctgacagtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctggtcaagggcttctacccttccgatatcgccgtggaatgggagagcaatggccagccagagaacaactacaagacaacccctcctgtgctggacagcgacggctcattcttcctgtacagcaagctgaccgtggacaagtccagatggcagcagggcaacgtgttcagctgcagcgtgatgcacgaggccctgcacaaccactacacccagaagtccctgtctctgagccccggcaaa

SEQ ID NO: 49 - DNA, 2979 bp, synthetic construct
  source 1..2979: mol_type = other DNA
gagatccagctggtgcagtctggcggaggactggctcagcctggcggctctatcagactgagctgtgcccccagcggctacatcagcagcgaccagatcctgaactgggtcaagaaggcccctggcaagtgcctggaatggatcggcagaatctaccccgtgaccggcgtgacccagtacaaccacaagttcgtgggcaaggccaccttcagcgtggacagatccaaggacaccgtgtacatgcagatgaacagcctgagagccgaggacaccggcgtgtactactgcggcagaggcgagacattcgacagctggggccagggcacactgctgaccgtgtcatctggtggaggcggtagcggcggaggaggatccggcggtggcggaagcgacatccagctgacccagagcatcagcagcctgagcgtgtccgtgggcgacagagtgaccatcaactgcaagagcaaccagaacctgctgtggagcggcaacagacggtacaccctcgtgtggcaccagtggaagcctggcaagagccccaagcccctgatcacatgggccagcgacagatccttcggcgtgcccagcagatttagcggcagcggctctgtgaccgacttcaccctgaccatcagctccgtgcagcccgaggacttcgccgtgtacttctgccagcagcacctggacatcccttacaccttcggctgcggcaccaagctggaaatcaagagaaccaccaccccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctaagcggggaagcggagagggcagaggatccctgctcacctgcggcgacgtggaggagaatcctggacctatggattttcaggtgcagattatcagcttcctgctaatcagtgcttcagtcataatgtctagagggggcgaggtccagctgcaagagtctggccctggactgaccaagcctagccagacactgagcctgacctgtagcgtgaccggctacagcatcaccagcgactactgggactggatcagaaagttccccggcaacaagctcgagtacatgggctacatcagctacaccggcagcacctactacaaccccagcctgagaagccggatctccatcaccagagacaccagcaagaaccagtactacctgcagctgagcagcgtgaccagcgaggatagcgccacctactattgcgctagaagaggcggctggctgagcccctttgtgtattggggacagggcaccctggtcacagtttctgctggtggcggaggatctggcggaggtggaagcggcggaggcggatctgatacagtgatgagccagtctcctagcagcctgggagtgtctgtgggcgagagagtgacactgacctgcaagagcagacagagcctgctgttcagcagcaaccagaagaacagcctggcctggtatcagcagaagcccggacagtctcccaagctgctgatctactgggccagcaccagagaaagcggcgtgcccgatagattcacaggctctggcagcggcaccgacttcaccctgacaatcagctccgtgaagtccgaggacctggccgtgtactactgccagcagtactacacataccccttcaccttcggagccggcaccagcctggaactgaaacttggcggaggaggcagcgacaagacccacacctgtcctccatgccctgcccctgagctgctgggcggaccctccgtgttcctgttccctccaaagcctaaggacaccctgatgatctcccggacccctgaagtgacctgcgtggtggtggacgtgtcccacgaggaccctgaagtgaagttcaattggtacgtggacggcgtggaagtgcacaacgccaagaccaagcctagagaggaacagtacgccagcacctacagagtggtgtccgtgctgacagtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctggtcaagggcttctacccttccgatatcgccgtggaatgggagagcaatggccagccagagaacaactacaagacaacccctcctgtgctggacagcgacggctcattcttcctgtacagcaagctgaccgtggacaagtccagatggcagcagggcaacgtgttcagctgcagcgtgatgcacgaggccctgcacaaccactacacccagaagtccctgtctctgagccccggcaaa

SEQ ID NO: 50 - DNA, 63 bp, synthetic construct
  source 1..63: mol_type = other DNA
atggccttaccagtgaccgccttgctcctgccgctggccttgctgctccacgccgccaggccg

SEQ ID NO: 51 - protein, 15 aa, synthetic construct
GGGGSGGGGSGGGGS